SEQUENCE LISTING (7 sequences)

SEQ ID NO: 1 - protein, 9 aa, Artificial Sequence
  first VH CDR
GYSITSDYA

SEQ ID NO: 2 - protein, 7 aa, Artificial Sequence
  second VH CDR
ISYSGST

SEQ ID NO: 3 - protein, 9 aa, Artificial Sequence
  third VH CDR
ARGGTYFDY

SEQ ID NO: 4 - protein, 6 aa, Artificial Sequence
  first VL CDR
AHINNW

SEQ ID NO: 5 - protein, 3 aa, Artificial Sequence
  second VL CDR
DAT

SEQ ID NO: 6 - protein, 9 aa, Artificial Sequence
  third VL CDR
QHYWSTPWT

SEQ ID NO: 7 - protein, 10 aa, Artificial Sequence
  Tag epitope I peptide
SAINNYAQKL

(19) United States
(12) Patent Application Publication
(10) Pub. No.: US 2019/0185571 A1
(43) Pub. Date: Jun. 20, 2019

(54) METHODS AND COMPOSITIONS FOR THE TREATMENT OF CANCER COMBINING AN ANTI-SMIC ANTIBODY AND IMMUNE CHECKPOINT INHIBITORS

(71) Applicant: MUSC FOUNDATION FOR RESEARCH DEVELOPMENT, Charleston, SC (US)

(72) Inventor: Jennifer WU, Charleston, SC (US)

(73) Assignee: MUSC FOUNDATION FOR RESEARCH DEVELOPMENT, Charleston, SC (US)

(21) Appl. No.: 16/318,928

(22) PCT Filed: Jul. 27, 2017
(86) PCT No.: PCT/US17/44078

§ 371 Date: Jan. 18, 2019

Related U.S. Application Data
(60) Provisional application No. 62/367,673, filed on Jul. 28, 2016.
(60) Provisional application No. 62/501,411, filed on May 4, 2017.

(51) Int. Cl. C07K 16/28; A61P 35/00; A61K 45/06; A61K 39/395; G01N 33/68

(52) U.S. Cl. CPC C07K 16/2833; C07K 16/2818; A61P 35/00; A61K 2039/507; A61K 39/3955; G01N 33/6854; A61K 45/06

(57) ABSTRACT

Provided herein are methods and compositions for the treatment of cancer comprising the combined administration of an anti-sMIC antibody and an immune checkpoint inhibitor. Methods and compositions for treating colitis with antibodies are also provided. Also provided herein are methods of predicting a response to an anti-sMIC antibody or immune checkpoint inhibitor therapy by measuring the level of serum sMIC in a subject.

This application claims the benefit of United States Provisional Patent Application Nos. 62/367,673, filed Jul. 28, 2016, and 62/501,411, filed May 4, 2017, the entirety of which are incorporated herein by reference.

The invention was made with government support under Grant No. 1R01 CA149405 awarded by the National Cancer Institute. The government has certain rights in the invention.

The sequence listing that is contained in the file named “MESC_P0099WO_ST25.txt”, which is 2 KB (as measured in Microsoft Windows) and was created on Jul. 25, 2017, is filed herewith by electronic submission and is incorporated by reference herein.

### BACKGROUND OF THE INVENTION

### 1. Field of the Invention

The present invention relates generally to the field of medicine and immunology. More particularly, it concerns methods and compositions for the treatment of cancer using combination therapies.

### 2. Description of Related Art

Blocking cytotoxic T lymphocyte antigen-4 (CTLA4) therapy improves the survival of patients with advanced melanoma and is in clinical trial for other cancer types (Wei et al., 2016; Wolchok et al., 2013a), such as prostate cancer, breast cancer, non-small cell lung cancer, lymphoma, and leukemia. Although an anti-CTLA4 antibody, ipilimumab, has been approved by the FDA for treatment of metastatic melanoma, its efficacy as a stand-alone therapy is limited to about a 15% overall response rate. Notably, anti-CTLA4 therapy has been associated with a significant risk of autoimmune toxicity, attributed to inflammation and breaking of self-tolerance in multiple organs. For example, in a Phase III clinical trial of ipilimumab in patients with metastatic melanoma, approximately 60% of patients demonstrated immune-related adverse events and 7 patients (1.1%) died from immune related adverse events (IRAEs) (Hodi et al., 2010). Combination therapy of anti-PD-1 and anti-CTLA4 has achieved greater overall response than monotherapy in patients with melanoma, the most immunogenic cancer; however, response is still limited to a subgroup of patients and is accompanied with increased toxicity (Larkin et al., 2015; Tsai and Daud, 2015; Wolchok et al., 2013b). Therefore, it is pivotal to develop a more effective combination therapy to increase the anti-tumor responses of immune checkpoint inhibitors, such as anti-CTLA4, in patients and simultaneously reduce the toxicity.

### SUMMARY OF THE INVENTION

A first embodiment of the present disclosure provides a method of treating cancer in a subject comprising administering an effective amount of: (a) an anti-soluble MIC (sMIC) antibody or antigen-binding fragment thereof and (b) at least one immune checkpoint inhibitor to the subject.

In some aspects, the anti-sMIC antibody or antigen-binding fragment thereof is a monoclonal antibody, a chimeric antibody, a CDR-grafted antibody, a humanized antibody, a Fab, a Fab′, a F(ab′)2, a Fv, or a scFv. In particular aspects, the anti-sMIC antibody is a monoclonal antibody. In specific aspects, the monoclonal antibody is B10G5 monoclonal antibody. In still further aspects, an antibody use according to the embodiments comprises a first VH CDR at least about 80%, 85%, 90% or 95% identical to SEQ ID NO: 1 (GYSITSDYA), a second VH CDR at least about 80%, 85%, 90% or 95% identical to SEQ ID NO: 2 (ISYSGST), a third VH CDR at least about 80%, 85%, 90% or 95% identical to SEQ ID NO: 3 (ARGGTYFDY), a first VL CDR at least about 80%, 85%, 90% or 95% identical to SEQ ID NO: 4 (AHINNW), a second VL CDR at least about 80%, 85%, 90% or 95% identical to SEQ ID NO: 5 (DAT), and a third VL CDR at least about 80%, 85%, 90% or 95% identical to SEQ ID NO: 6 (QHYWSTPWT). In yet further aspects, an antibody use according to the embodiments comprises a first VH CDR identical to SEQ ID NO: 1, a second VH CDR identical to SEQ ID NO: 2, a third VH CDR identical to SEQ ID NO: 3, a first VL CDR identical to SEQ ID NO: 4, a second VL CDR identical to SEQ ID NO: 5, and a third VL CDR identical to SEQ ID NO: 6. In some aspects, the antibody is a humanized antibody comprising a first VH CDR identical to SEQ ID NO: 1, a second VH CDR identical to SEQ ID NO: 2, a third VH CDR identical to SEQ ID NO: 3, a first VL CDR identical to SEQ ID NO: 4, a second VL CDR identical to SEQ ID NO: 5, and a third VL CDR identical to SEQ ID NO: 6.

In certain aspects, the at least one immune checkpoint inhibitor is selected from an inhibitor of CTLA-4, PD-1, PD-L1, PD-L2, LAG-3, BTLA, B7H3, B7H4, TIM3, KIR, or A2aR. In some aspects, the at least one immune checkpoint inhibitor is a human programmed cell death 1 (PD-1) axis binding antagonist. In further aspects, the PD-1 axis binding antagonist is selected from the group consisting of a PD-1 binding antagonist, a PDL1 binding antagonist and a PDL2 binding antagonist. In certain aspects, the PD-1 axis binding antagonist is a PD-1 binding antagonist. In some aspects, the PD-1 binding antagonist inhibits the binding of PD-1 to PDL1 and/or PDL2. In particular aspects, the PD-1 binding antagonist is a monoclonal antibody or antigen binding fragment thereof. In specific aspects, the PD-1 binding antagonist is nivolumab, pembrolizumab, pidillizumab, AMP-514, REGN2810, CT-011, BMS 936559, MPDL328OA or AMP-224. In other aspects, the at least one immune checkpoint inhibitor is an anti-CTLA-4 antibody. In particular aspects, the anti-CTLA-4 antibody is tremelimumab or ipilimumab. In further aspects, the at least one immune checkpoint inhibitor is an anti-killer-cell immunoglobulin-like receptor (KIR) antibody. In specific aspects, the anti-KIR antibody is lirilumab. In some aspects, two or three immune checkpoint are administered in combination with the anti-sMIC antibody or antigen-binding fragment thereof. In some aspects, the two immune checkpoint inhibitors comprise an anti-PD1 antibody and an anti-CTLA4 antibody.

In additional aspects, the immune checkpoint inhibitor is a recombinant oncolytic virus. In some aspects, the recombinant oncolytic virus comprises an expression cassette encoding a soluble form of PD1. In certain aspects, the virus is replication competent. In some aspects, the expression cassette is under the control of a viral promoter. In particular aspects, the viral promoter is synthetic early/late poxvirus promoter. In some aspects, the virus is selected from the group consisting of myxoma virus, reovirus, herpes simplex virus, Newcastle Disease virus, measles virus, retrovirus, poxvirus, rhabdovirus, picornavirus, coxsackievirus and parvovirus. In specific aspects, the oncolytic virus is myxoma virus.

In certain aspects, the cancer is melanoma, non-small cell lung, small-cell lung, lung, hepatocarcinoma, retinoblastoma, astrocytoma, glioblastoma, leukemia, neuroblastoma, head, neck, breast, pancreatic, prostate, renal, bone, testicular, ovarian, mesothelioma, cervical, gastrointestinal, urogenital, respiratory tract, hematopoietic, musculoskeletal, neuroendocrine, carcinoma, sarcoma, central nervous system, peripheral nervous system, lymphoma, brain, colon or bladder cancer. In particular aspects, the cancer is prostate cancer. In specific aspects, the prostate cancer is further defined as castration-resistant prostate cancer.

In some aspects, the cancer is a MIC-positive cancer. In certain aspects, the subject has a high serum level of sMIC. In particular aspects, the high serum level of sMIC is at least 0.5 ng/mL, such as at least 1, 2, 3, 4, or 5 ng/mL. In some aspects, the serum level of sMIC in the subject decreases after administration of the combination therapy, such as an at least 2-fold decrease.

In further aspects, the combination therapy of an anti-sMIC antibody or antigen-binding fragment thereof and at least one immune checkpoint inhibitor results in an increase in CD8⁺ T cells, such as an at least 2-fold increase in the percentage of CD8⁺ T cells. In some aspects, the increase in CD8⁺ T cells is specific to the tumor draining lymph nodes (dLN), spleen, and/or tumor infiltrates.

In certain aspects, the anti-sMIC antibody or antigen-binding fragment thereof and at least one immune checkpoint inhibitor results in increased NKG2D expression on CD8⁺ T cells. In additional aspects, the anti-sMIC antibody or antigen-binding fragment thereof and at least one immune checkpoint inhibitor results in an increase in effector memory-like CD44^{hi} CD8⁺ T cells in the spleen, tumor dLN, and/or tumor infiltrates. In some aspects, the combination therapy results in an increase in IFNγ production.

In additional aspects, the method further comprises the adoptive transfer of T cells. In some aspects, the T cells are CD8⁺ T cells. In some aspects, the CD8⁺ T cells are antigen-specific CD8⁺ T cells. In some aspects, the anti-sMIC antibody and at least one immune checkpoint inhibitor enhance the sustainability of the T cells in the subject.

In some aspects, the combination therapy enhances dendritic cell activation. In certain aspects, dendritic cell activation comprises an increase in the expression of co-stimulatory molecule CD80 and/or CD86. In other aspects, the combination therapy increases TCR clonality and/or repertoire diversity. In some aspects, combination therapy increases the intracellular levels of CD3ζ in CD8⁺ T cells in the subject.

In certain aspects, the anti-sMIC antibody and/or at least one immune checkpoint inhibitor is administered intratumorally, intraarterially, intravenously, intravascularly, intrapleuraly, intraperitoneally, intratracheally, intrathecally, intramuscularly, endoscopically, intralesionally, percutaneously, subcutaneously, regionally, stereotactically, or by direct injection or perfusion. In some aspects, the anti-sMIC antibody is administered prior to the immune checkpoint inhibitor. In other aspects, the anti-sMIC antibody is administered simultaneously with or after the immune checkpoint inhibitor.

In further aspects, more than one checkpoint inhibitor is administered, such as 2, 3, or 4 immune checkpoint inhibitors. In some aspects, the anti-sMIC antibody and/or immune checkpoint inhibitor(s) are administered more than once, such as 2, 3, or 4 times, such as daily, weekly, or monthly.

In additional aspects, the method further comprises administering at least one additional anticancer treatment. In some aspects, the at least one additional anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy. In particular aspects, the biological therapy is a monoclonal antibody, siRNA, miRNA, antisense oligonucleotide, ribozyme or gene therapy.

A further embodiment provides a pharmaceutical composition comprising an anti-sMIC antibody and at least one immune checkpoint inhibitor. Another embodiments provides a composition comprising an effective amount of an anti-sMIC antibody or antigen-binding fragment thereof and at least one immune checkpoint inhibitor for use in the treatment of cancer in a subject.

In some aspects of the above embodiments, the anti-sMIC antibody is a monoclonal antibody, a chimeric antibody, a CDR-grafted antibody, a humanized antibody, a Fab, a Fab′, a F(ab′)2, a Fv, or a scFv. In particular aspects, the anti-sMIC antibody is a monoclonal antibody. In specific aspects, the monoclonal antibody is B10G5 monoclonal antibody. In certain aspects, the at least one immune checkpoint inhibitor is an anti-PD1 antibody and/or anti-CTLA4 antibody. In particular aspects, the at least immune checkpoint inhibitor is comprised in an oncolytic virus. In specific aspects, the oncolytic virus is myxoma virus. In one particular aspect, the myxoma virus expresses soluble PD-1.

An even further embodiment provides a method of predicting a response to an immune checkpoint inhibitor in a patient having a cancer comprising detecting a level of serum sMIC in a sample obtained from said patient, wherein if the serum sMIC level is low, then the patient is predicted to have a favorable response to the immune checkpoint inhibitor. In some aspects, a low serum sMIC level is further defined as less than 0.1, 0.2, 0.3, 0.4, or 0.5 ng/mL. In certain aspects, a low serum sMIC level comprises a level between 0-0.1, 0.1-0.3, 0.2-0.4, 0.3-0.5, 0.5-0.8, 0.6-1, or 1-1.5 ng/mL. In additional aspects, if the level of serum sMIC is high, then the patient is predicted to have adverse reaction to the immune checkpoint inhibitor. In particular aspects, a high serum sMIC level identifies a patient in need of an anti-sMIC antibody in combination with the immune checkpoint inhibitor.

In another embodiment, there is provided a method of treating colitis in a subject comprising administering an effective amount of an anti-sMIC antibody or antigen-binding fragment thereof to the subject. In some aspects, the anti-sMIC antibody or antigen-binding fragment thereof is a monoclonal antibody, a chimeric antibody, a CDR-grafted antibody, a humanized antibody, a Fab, a Fab′, a F(ab′)2, a Fv, or a scFv. In certain aspects, the anti-sMIC antibody is a monoclonal antibody. In one specific aspect, the monoclonal antibody is B10G5 monoclonal antibody.

In some aspects, the colitis is infectious colitis, inflammatory bowel disease (IBD), ischemic colitis, diversion colitis, chemical colitis, or microscopic colitis. In particular aspects, the IBD is Crohn's disease or ulcerative colitis.

In certain aspects, the subject has a high serum level of sMIC. In some aspects, the high serum level of sMIC is at least 0.5 ng/mL. In some aspects, the serum level of sMIC in the subject decreases after administration of the anti-sMIC antibody.

In some aspects, the anti-sMIC antibody is administered intratumorally, intraarterially, intravenously, intravascularly, intrapleuraly, intraperitoneally, intratracheally, intrathecally, intramuscularly, endoscopically, intralesionally, percutaneously, subcutaneously, regionally, stereotactically, or by direct injection or perfusion. In particular aspects, the anti-sMIC antibody is administered subcutaneously.

In additional aspects, the method further comprises administering at least one additional therapy. In some aspects, the at least one additional therapy is an aminosalicylate, a corticosteroid, and/or an immunosuppressant. In particular aspects, the aminosalicylate is sulfasalazine, mesalazine, balsalazide, and/or olsalazine. In certain aspects, the corticosteroid is prednisone and/or hydrocortisone. In specific aspects, the immunosuppressant is azathioprine, mercaptopurine, cyclosporingm infliximab, adalimumab, golimumab, and/or vedolizumab.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-F: Mice with high circulating tumor shed soluble MIC (sMIC) presented adverse response to anti-CTLA4 therapy in a clinically relevant TRAMP/MICB spontaneous tumor model. TRAMP or TRAMP/MICB mice models had tumor initiation at 16 weeks and metastasis at 24 weeks (A). The adverse response was exemplified by reduced overall survival (B), shortened median survival time, and more lung metastasis (D-E). (F) Level of sMIC in control TRAMP mice and TRAMP/MICB survivors and non-survivors.

FIGS. 2A-E: Antibody B10G5 targeting sMIC remarkably enhances response to anti-CTLA4 therapy. (A) Schematic of therapy timeline for TRAMP/MICB mice. (B) Survival curve of treatment with anti-CTLA4 and combination with anti-sMIC. (C) Serum sMIC at necroposy of TRAMP/MICB mice at 36 weeks. (D) Weight of primary tumors at 36 weeks. (E) Immunohistochemistry of cleaved caspase 3 and CD8 demonstrating that combination therapy effectively induces tumor cell apoptosis (cleaved caspase 3) and induces infiltration of CD8⁺ T cells in tumor parenchyma.

FIGS. 3A-H: Combination therapy of anti-sMIC and anti-CTLA4 remarkably increase CD8⁺ T cell population and function in tumor draining lymph nodes and tumor beds. (A,C, E) Representative graphs of flow cytometry analyses demonstrating increased percentage of CD8⁺ T cells, NKG2D expression on CD8⁺ T cell and CD44⁺ CD8⁺ T cell population. (B,D,G) Summary data. (F,H) IFNγ production by CD8⁺ T cells in response to ex vivo stimulation with PMA and Ionomycin.

FIGS. 4A-H: Combination therapy of anti-sMIC and anti-CTLA4 cooperatively enhance antigen-specific CD8 T cell anti-tumor responses. Tumor antigen SV40Tag-specific TCR-I CD8⁺ T cells were transferred into mice. Data shown are 14 days post-transfer of number of SV40Tag-specific CD8⁺ T cells (D^{b}/I-tetramer positive, (A, B), proliferation of SV40Tag-specific CD8⁺ T cells (CFSE^{lo} of D^{b}/I-tetramer⁺ CD8 T cells, (C, D)), IFNg production by D^{b}/I-tetramer⁺ CD8⁺ T cells in response to PMA/I stimulation (E, F), and activation of D^{b}/I-tetramer⁺ CD8⁺ T cells (CD44⁺%, (G, H)).

FIGS. 5A-B: B10G5 and anto-CTLA4 therapy cooperatively increase DC activation (CD40) and the expression of co-stimulatory molecules CD80/86. (A) Representative histograms from flow cytometry analyses. Gray filled profiles, control isotype staining. Open dark profiles, antibody to specific DC surface molecules. (B) Summary data.

FIGS. 6A-C: B10G5 therapy increase T cell clonality and TCR complexity in tumors. DNA were isolated from paraffin embedded tumor sections and subjected to ImmunoSEQ assay (Adaptive Biotechnologies). Productive TCR template (A), TCR clonality (B), and complexity (C) were analyzed using the Shannon diversity index using ImmunoSEQ Analyzer software.

FIG. 7: Neutralizing sMIC stabilizing TCR signaling molecule TCR3ζ. Mouse CD8⁺ T cells sere stimulated with plate-bund anti-CD3 antibody and soluble antio-CD28 antibody, in the presence of sMIC or sMIC and anti-sMIC mAb B10G5 for 3 days. Cell lysates were subjected for Western Blot analyses with anti-CD3ζ antibody. anti-β-actin, loading control.

FIGS. 8A-E: Induction of subclinical colitis in sMIC^{hi} TRAMP/MIC mice in response to anti-CTLA4 (αCTLA4) therapy. (A) Representative gross morphology of colon. (B) Summary of colon length. *, p<0.05 (C) Representative histology of colon. (D) Representative RT-PCR demonstrating tissue specific MICB expression in male MICB/B6 or TRAMP/MICB transgenic mice; no MICB expression was detected in colon. (E) Representative histology demonstrating normal colon histology in male MICB/B6 transgenic mice.

FIGS. 9A-C: Recapitulation of the negative effect of sMIC on anti-CTLA4 therapy in transplantable tumor model. (A) Schematic depiction of the transplantable tumor model and therapy. (B) Tumor growth curve. (C) Representative histology of the colon.

FIGS. 10A-B: Antibody neutralizing sMIC eliminates colitis in TRAMP/MICB mice that received anti-CTLA4 (αCTLA4) therapy. (A) Summary of colon length. (B) Representative histology of the colon.

FIG. 11: Detection of anti-sMIC autoantibody in the sera of a small cohort of prostate cancer patients who has metastatic disease and enrolled in a clinical trial (NCT01498978) of ipilimumab in combination with hormone suppression at Knight Cancer Center. The study of South Carolina as the sample coded and received de-ID'ed for ELISA assay. Serum autoantibody was detected by direct ELISA against antigen sMIC and anti-human Fc as detecting agent. Patient 5254-8 presented durable response as measured by serum PSA.

FIGS. 12A-D: Marginal response of CD8 T cells in TRAMP mice to anti-CTLA4 therapy. Data representatively show flow cytometry graphs of CD8 T cell population (A), NKG2D expression on CD8 T cells (B), IFNγ production in response to ex vivo PMA/I restimulation (C), and memory-like/activation status (D). Spin, spleen. dLN, tumor-draining lymph nodes. TIL, tumor infiltrates. cIgC, control IgG.

FIGS. 13A-B: Circulating sMIC or anti-sMIC autoantibody impacts response to anti-CTLA4 therapy in in TRAMP/MICB mice in comparison to MIC-negative TRAMP mice. Data shown are representatives of NK and CD8 T cell responses in tumor draining lymph nodes. (A) High levels of circulating sMIC reduce CD8 T function whereas autoantibody revamps the function of CD8 T cells in response to anti-CTLA4 in TRAMP/MICB mice. The impact on the number of CD8 T cells I marginal. (B) High levels of circulating sMIC reduce NK cell number and function, whereas autoantibody revamps the number and function of NK cells in response to anti-CTLA4 therapy.

FIGS. 14A-B: Therapy has no effect on the activation or co-stimulatory molecule on DCs in spleen or non-tumor draining LN. (A) Flow cytometry of CD11c and MHCII. (B) Summary data of the mean fluorescence intensity (MFI) of respective molecules.

FIGS. 15A-B: B10G5 significantly enhanced therapeutic efficacy of immune checkpoint Blockade anti-PD-L1 (A) and anti-CTLA4 (B) in preclinical MIC⁺ TRAMP/MIC mice (˜27-wks old) which have developed advanced prostate carcinoma. Data shown are final prostate mass of survived animals with 8-wk treatment. Larger cohorts of mice are currently enrolled in the combined therapy of B10G5 and anti-CTLA4.

FIG. 16: B10G5 and anti-PD-L1 antibody cooperatively inhibit tumor growth of B16-sMICB transplantable tumor. B16-sMICB cells were implanted into B6/MICB mice. Therapy initiated when tumor volume reaches approximate 100 mm³. Kaplan-Meier survival curve. Tumor volume of 1200 mm³ was defined as survival end point. *p<0.01 in comparison to anti-PDL1 or B10G5 monotherapy. N=10 per group. The combination B10G5 and anti-PD-L1 therapy resulted in the highest percentage survival. The single B10G5 and anti-PD-L1 therapies had similar percentage survival while the control cIgGs had the lowest percentage survival.

### DESCRIPTION OF ILLUSTRATIVE EMBODIMENTS

Effective anti-tumor immune response not only requires elimination of co-inhibitory signals, but also requires provision of co-stimulatory signals on effector T cells. NKG2D, the NK cell activating receptor has been shown to provide unique non-redundant costimulation from CD28 to antigen-specific CD8 T cells (Bauer et al., 1999; Groh et al., 2001; Markiewicz et al., 2005; Serrano-Pertierra et al., 2014; Roberts et al., 2001). In cancer patients, NKG2D on effector T cells is often down-modulated by soluble NKG2D ligands, among which the most common member is the MHC I chain-related molecule (MIC) (Groh et al., 2002; Marten et al., 2006; Wang et al., 2008; Holdenrieder et al., 2006a; Holdenrieder et al., 2006b; Salih et al., 2008; Salih et al., 2003). Tumor-derived sMIC also suppresses host immune response by perturbing NK cell homeostatic maintenance and function, driving the expansion of myeloid suppressor cells and skewing macrophage into alternatively activated phenotype (Liu et al., 2013; Wu et al., 2004; Xiao et al., 2015).

Accordingly, in certain embodiments, the present disclosure provides methods and compositions for the treatment of cancer by targeting sMIC in combination with at least one immune checkpoint inhibitor. In the present studies, a clinically relevant bi-transgenic mouse model that closely recapitulates the onco-immune dynamic of human cancer was used to demonstrate the effectiveness of therapy of targeting sMIC, such as with a sMIC-neutralizing antibody B10G5, and an immune checkpoint inhibitor to abrogate the immune suppressive effect of sMIC and overcome immune tolerance of antigen-specific CD8⁺ T cells. Indeed, co-administration of a sMIC-neutralizing antibody not only alleviated the adverse response, but also enhanced the response to immune checkpoint inhibitors, such as anti-CTLA4 and anti-PD1.

In addition, it was observed that sMIC^{hi} animals also developed colitis in response to anti-CTLA4 therapy. Thus, methods are also provided herein for the treatment of colitis by the administration of a sMIC-neutralizing antibody.

Further, it was observed that subjects with elevated serum sMIC had a severe adverse response to anti-CTLA4 therapy and significantly shortened survival due to worsened diseases. Thus, methods are also provided herein to select for patients who will have a favorable response to immune checkpoint inhibitor therapy, such as an anti-CTLA4 or anti-PD1 antibody, by measuring the level of serum sMIC.

### I. DEFINITIONS

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

As used herein “soluble MIC” or “sMIC” refers to a portion of a MIC polypeptide that is lacking a transmembrane domain, e.g. an extracellular portion of MIC that has been cleaved from the transmembrane domain.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated.” The terms “purified” or “substantially purified” refer to an isolated nucleic acid or polypeptide that is at least 95% by weight the subject nucleic acid or polypeptide, including, for example, at least 96%, at least 97%, at least 98%, at least 99% or more.

As used herein, an “epitope” can be formed on a polypeptide both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin VH/VL pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics.

Accordingly, as used herein, “selectively binds” or “specifically binds” refers to the ability of an anti-MIC-binding polypeptide (e.g., an antibody or portion thereof) described herein to bind to a target, such as a MIC molecule present on the cell-surface, with a KD 10^{˜5} M (10000 nM) or less, e.g., 10^{˜6} M, 10^{˜7} M, 10^{˜8}M, 10^{˜9}M, 10^{˜10} M, 10^{˜U} M, 10^{˜12} M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay. A polypeptide specifically bound to a target is not displaced by a non-similar competitor. In certain embodiments, an antibody or antigen-binding portion thereof is said to specifically bind an antigen when it preferentially recognizes its target antigen in a complex mixture of proteins and/or macromolecules.

As used herein, the terms “proteins” and “polypeptides” are used interchangeably herein to designate a series of amino acid residues each connected to the other by peptide bonds between the alpha-amino and carboxy groups of adjacent residues. The polymer of protein amino acids, including modified amino acids (e.g., phosphorylated, glycated, or glycosylated) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “antibody” refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and antigen-binding portions thereof; including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding fragment thereof, bifunctional hybrid antibodies (e.g., Lanzavecchia et al., 1987) and single chains (e.g., Huston et al., 1988; Bird et al., 1988), which are incorporated herein by reference).

The terms “antigen-binding fragment” or “antigen-binding portion” of an antibody, used interchangeably herein, refer to one or more fragments of an antibody as described herein, said fragments still having the binding affinities as defined above herein. Fragments of a complete antibody have been shown to be able to carry out the antigen-binding function of an antibody. In accordance with the term “antigen-binding portion” of an antibody, examples of binding fragments include (i) an Fab fragment, i.e. a monovalent fragment composed of the VL, VH, CL and CHI domains; (ii) an F(ab′)2 fragment, i.e. a bivalent fragment comprising two Fab fragments linked to one another in the hinge region via a disulfide bridge; (iii) an Fd fragment composed of the VH and CHI domains; (iv) an Fv fragment composed of the FL and VH domains of a single arm of an antibody; and (v) a dAb fragment (Ward et al., 1989) consisting of a VH domain or of VH, CHI, CH2, DH3, or VH, CH2, CH3 (dAbs, or single domain antibodies, comprising only VL domains have also been shown to specifically bind to target eptiopes). Although the two domains of the Fv fragment, namely VL and VH, are encoded by separate genes, they may further be linked to one another using a synthetic linker, e.g. a poly-G4S amino acid sequence, and recombinant methods, making it possible to prepare them as a single protein chain in which the VL and VH regions combine in order to form monovalent molecules (known as single chain Fv (ScFv); see, for example, Bird et al., 1988; Huston et al., 1988). The term “antigen-binding portion” of an antibody is also intended to comprise such single chain antibodies. Other forms of single chain antibodies such as “diabodies” are likewise included here. Diabodies are bivalent, bispecific antibodies in which VH and VL domains are expressed on a single polypeptide chain, but using a linker which is too short for the two domains being able to combine on the same chain, thereby forcing said domains to pair with complementary domains of a different chain and to form two antigen-binding sites (see, for example, Holliger et al., 1993; Poljak et al., 1994). An immunoglobulin constant domain refers to a heavy or light chain constant domain. Human IgG heavy chain and light chain constant domain amino acid sequences are known in the art.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The term “human antibody” or “humanized antibody” refers to antibodies whose variable and constant regions correspond to or are derived from immunoglobulin sequences of the human germ line, as described, for example, by Kabat et al. (see Kabat, et al., 1991). However, the human antibodies can contain amino acid residues not encoded by human germ line immunoglobulin sequences (for example mutations which have been introduced by random or site-specific mutagenesis in vitro or by somatic mutation in vivo), for example in the CDRs, and in particular in CDR3. Recombinant human antibodies as described herein have variable regions and may also contain constant regions derived from immunoglobulin sequences of the human germ line (see Kabat, et al., 1991). According to particular embodiments, however, such recombinant human antibodies are subjected to in vitro mutagenesis (or to a somatic in vivo mutagenesis, if an animal is used which is transgenic due to human Ig sequences) so that the amino acid sequences of the VH and VL regions of the recombinant antibodies are sequences which although related to or derived from VH and VL sequences of the human germ line, do not naturally exist in vivo within the human antibody germ line repertoire. According to particular embodiments, recombinant antibodies of this kind are the result of selective mutagenesis or back mutation or of both. Preferably, mutagenesis leads to an affinity to the target which is greater, and/or an affinity to non-target structures which is smaller than that of the parent antibody.

The term “chimeric antibody” refers to antibodies which contain sequences for the variable region of the heavy and light chains from one species and constant region sequences from another species, such as antibodies having murine heavy and light chain variable regions linked to human constant regions. Humanized antibodies have variable region framework residues substantially from a human antibody (termed an acceptor antibody) and complementarity determining regions substantially from a non-human antibody, e.g. a mouse-antibody, (referred to as the donor immunoglobulin). See, Queen et al., 1989, WO 90/07861, U.S. Pat. Nos. 5,693,762, 5,693,761, 5,585,089, 5,530,101 and 5,225,539, which are herein incorporated by reference in their entirety. The constant region(s), if present, are also substantially or entirely from a human immunoglobulin. The human variable domains are usually chosen from human antibodies whose framework sequences exhibit a high degree of sequence identity with the (murine) variable region domains from which the CDRs were derived. The heavy and light chain variable region framework residues can be substantially similar to a region of the same or different human antibody sequences. The human antibody sequences can be the sequences of naturally occurring human antibodies or can be consensus sequences of several human antibodies. See Carter et al., WO 92/22653, which is herein incorporated by reference in its entirety.

As used herein, the phrase “therapeutically effective amount”, “effective amount” or “effective dose” refers to an amount that provides a therapeutic or aesthetic benefit in the treatment, prevention, or management of a tumor or malignancy, e.g. an amount that provides a statistically significant decrease in at least one symptom, sign, or marker of a tumor or malignancy. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The terms “decrease,” “reduce,” “reduced”, “reduction”, “decrease,” and “inhibit” are all used herein generally to mean a decrease by a statistically significant amount relative to a reference. However, for avoidance of doubt, “reduce,” “reduction” or “decrease” or “inhibit” typically means a decrease by at least 10% as compared to a reference level and can include, for example, a decrease by at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, up to and including, for example, the complete absence of the given entity or parameter as compared to the reference level, or any decrease between 10-99% as compared to the absence of a given treatment.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

A “tumor” as used herein refers to an uncontrolled growth of cells which interferes with the normal functioning of the bodily organs and systems. The terms “cancer” and “malignancy” refer to a tumor that is metastatic, i.e. that is it has become invasive, seeding tumor growth in tissues remote from the original tumor site. A subject that has a cancer or a tumor is a subject having objectively measurable cancer cells present in the subject's body. Included in this definition are benign tumors and malignant cancers, as well as potentially dormant tumors or micrometastatses. Cancers which migrate from their original location and seed other vital organs can eventually lead to the death of the subject through the functional deterioration of the affected organs. Hemopoietic cancers, such as leukemia, are able to out-compete the normal hemopoietic compartments in a subject, thereby leading to hemopoietic failure (in the form of anemia, thrombocytopenia and neutropenia) ultimately causing death.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient”, “individual” and “subject” are used interchangeably herein.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a tumor or malignancy, delay or slowing of tumor growth and/or metastasis, and an increased lifespan as compared to that expected in the absence of treatment. As used herein, the term “administering,” refers to the placement of an antibody or antigen-binding portion thereof as described herein or a nucleic acid encoding an antibody or antigen-binding portion thereof as described herein into a subject by a method or route which results in at least partial localization of the agents at a desired site. The pharmaceutical composition comprising an antibody or antigen-binding portion thereof as described herein or a nucleic acid encoding an antibody or antigen-binding portion thereof as described herein disclosed herein can be administered by any appropriate route which results in an effective treatment in the subject.

As used herein, the terms “chemotherapy” or “chemotherapeutic agent” refer to any chemical agent with therapeutic usefulness in the treatment of diseases characterized by abnormal cell growth. Such diseases include tumors, neoplasms and cancer as well as diseases characterized by hyperplastic growth. Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most if not all of these agents are directly toxic to cancer cells and do not require immune stimulation. In one embodiment, a chemotherapeutic agent is an agent of use in treating neoplasms such as solid tumors. In one embodiment, a chemotherapeutic agent is a radioactive molecule. One of skill in the art can readily identify a chemotherapeutic agent of use (e.g. see Slapak and Kufe, Principles of Cancer Therapy; Perry et al., 2000). The bispecific and multispecific polypeptide agents described herein can be used in conjunction with additional chemotherapeutic agents.

As used herein, “immunotherapy” refers to a diverse set of therapeutic strategies designed to induce the patient's own immune system to fight the tumor, and include, but are not limited to, intravesical BCG immunotherapy for superficial bladder cancer, vaccines to generate specific immune responses, such as for malignant melanoma and renal cell carcinoma, the use of Sipuleucel-T for prostate cancer, in which dendritic cells from the patient are loaded with prostatic acid phosphatase peptides to induce a specific immune response against prostate-derived cells, administration of cytokines, growth factors and/or signaling molecules that stimulate one or more immune cell type (e.g. interleukins), ex vivo expansion and/or stimulation of lymphocytes and/or dendritic cell specific for a tumor antigen prior to reintroduction to the patient, imiquimod, adoptive cell transfer, and/or the methods described, e.g., in International Patent Publication WO 2003/063792 and U.S. Pat. No. 8,329,660. In some embodiments, the immunotherapy stimulates NK responses. In some embodiments, the immunotherapy is an adoptive cell transfer approach.

The term “immune checkpoint” refers to pathways in the immune system which provides inhibitory signals to its components in order to balance immune reactions. Known immune checkpoint proteins comprise CTLA-4, PD-1 and its ligands PD-L1 and PD-L2 and in addition LAG-3, BTLA, B7H3, B7H4, TIM3, MR. The pathways involving LAG3, BTLA, B7H3, B7H4, TIM3, and MR are recognized in the art to constitute immune checkpoint pathways similar to the CTLA-4 and PD-1 dependent pathways (see e.g. Pardoll, 2012; Mellman et al., 2011).

The term “PD-1 axis binding antagonist” refers to a molecule that inhibits the interaction of a PD-1 axis binding partner with either one or more of its binding partners, so as to remove T-cell dysfunction resulting from signaling on the PD-1 signaling axis—with a result being to restore or enhance T-cell function (e.g., proliferation, cytokine production, target cell killing). As used herein, a PD-1 axis binding antagonist includes a PD-1 binding antagonist, a PD-L1 binding antagonist and a PD-L2 binding antagonist.

The term “PD-1 binding antagonist” refers to a molecule that decreases, blocks, inhibits, abrogates or interferes with signal transduction resulting from the interaction of PD-1 with one or more of its binding partners, such as PD-L1 and/or PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is CT-011 (pidilizumab). In another specific aspect, a PD-1 binding antagonist is AMP-224.

The term “PD-L1 binding antagonist” refers to a molecule that decreases, blocks, inhibits, abrogates or interferes with signal transduction resulting from the interaction of PD-L1 with either one or more of its binding partners, such as PD-1 or B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1 or B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody. In a specific aspect, an anti-PD-L1 antibody is YW243.55.S70. In another specific aspect, an anti-PD-L1 antibody is MDX-1105. In still another specific aspect, an anti-PD-L1 antibody is MPDL3280A. In still another specific aspect, an anti-PD-L1 antibody is MEDI4736.

The term “PD-L2 binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to one or more of its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

An “immune checkpoint inhibitor” refers to any compound inhibiting the function of an immune checkpoint protein. Inhibition includes reduction of function and full blockade. In particular the immune checkpoint protein is a human immune checkpoint protein. Thus the immune checkpoint protein inhibitor in particular is an inhibitor of a human immune checkpoint protein.

The term “oncolytic virus,” as used herein, refers to a virus capable of selectively replicating in and slowing the growth or inducing the death of a cancerous or hyperproliferative cell, either in vitro or in vivo, while having no or minimal effect on normal cells. Exemplary oncolytic viruses include vesicular stomatitis virus (VSV), Newcastle disease virus (NDV), herpes simplex virus (HSV), reovirus, measles virus, retrovirus, influenza virus, Sinbis virus, vaccinia virus, and adenovirus.

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

### II. MIC NEUTRALIZING ANTIBODY

Certain embodiments of the present disclosure concern an antibody or antigen-binding portion thereof that specifically binds to soluble Major Histocompatibility Complex class I chain-related (sMIC). In some embodiments, the methods and compositions described herein concern inhibition of sMIC, e.g. reducing the level and/or activity of sMIC that is available to interact with cellular receptors. In some embodiments, inhibition of sMIC can be a reduction of unbound sMIC (e.g. sMIC not bound to a receptor and/or available to be bound by an antibody). In some embodiments, inhibition of sMIC can be a reduction of the level of sMIC, e.g. the level of sMIC in circulation.

MIC is a ligand on the surface of epithelial cells that binds to activating NKG2D receptors on the surface of Natural Killer (NK) cells. Activation of the NK cells through NKG2D mediated binding increases anti-tumor immunogenicity. These ligands are not normally present on cell surfaces, but can be stimulated through cytokine activation, i.e., an inflammatory response. Tumors expressing MIC are noted as MIC⁺ and are associated with poorer prognosis. When MIC is not present, the level of NKG2D receptors is unchanged on the NK cells. However, as the level of MIC increases, the level of NKG2D decreases, thus decreasing the potential for an immunological response to a tumor. The mechanism by which MIC expression impacts NKG2D expression is mediated by the shedding of MIC (aka, “soluble MIC”, “shedding-derived MIC”, “soluble NKG2D ligand”, and “sMIC”) from the surface of tumor cells.

MIC polypeptides are surface transmembrane proteins. The presence of a MIC polypeptide on the cell surface can signal the immune receptor NKG2D for tumor immune destruction, typically by NK cells and cytotoxic T cells (CTLs). However, in many tumors, MIC is shed from the tumor surface, resulting in decreased host immunity against the tumor cell and promoting tumor evasion and progression. MIC polypeptides include, but are not limited to the human MICA (e.g. NCBI Ref Seqs NP 000238 and 001 170990) and human MICB (e.g. NCBI Ref Seq: NP 005922). In some embodiments, a MIC polypeptide can comprise MICA. In some embodiments, a MIC polypeptide can comprise MICB.

In some aspects, targeting of sMIC comprises administration of an anti-sMIC antibody, such as a monoclonal antibody. One exemplary monoclonal anti-sMIC antibody is B10G5 (International Publication No. WO 2015/003114; incorporated herein by reference in its entirety), a sMIC neutralizing, but non-blocking anti-MIC monoclonal antibody. The B10G5 mAb neutralizes circulating sMIC (thus eliminating its antagonistic effect on the expression of NKG2D), but also recognizes tumor cell surface membrane-bound MIC, because sMIC shares the same sequence and structure as the ectodomain of cell-surface MIC. Because B10G5 and the receptor NKG2D recognize different epitopes of MIC, B10G5 does not block the binding of NKG2D to MIC ligands on tumor cells (i.e., the B10G5 and NKG2D bind to different sites on MIC).

Furthermore, an antibody as described herein or an antigen-binding portion thereof may be part of a larger immunoadhesion molecule formed by covalent or noncovalent association of said antibody or antibody portion with one or more further proteins or peptides. Relevant to such immunoadhesion molecules are the use of the streptavidin core region in order to prepare a tetrameric scFv molecule (Kipriyanov et al., 1995) and the use of a cystein residue, a marker peptide and a C-terminal polyhistidinyl, e.g. hexahistidinyl tag in order to produce bivalent and biotinylated scFv molecules (Kipriyanov et al., 1994).

In some embodiments, the antibody and/or antigen-binding portion thereof described herein can be an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, and a functionally active epitope-binding fragment thereof.

As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retain the ability to specifically bind the target antigen (e.g. an epitope present on sMIC) of a MIC polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as He, Val, Leu, or Ala for one another), or substitution of one polar residue for another (such as between Lys and Arg; Glu and Asp; or Gin and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), He (I), Pro (P), Phe (F), Trp (W), Met (M); (2) uncharged polar: Gly (G), Ser (S), Thr (T), Cys (C), Tyr (Y), Asn (N), Gin (Q); (3) acidic: Asp (D), Glu (E); (4) basic: Lys (K), Arg (R), His (H).

Alternatively, naturally occurring residues can be divided into groups based on common side-chain properties: (1) hydrophobic: Norleucine, Met, Ala, Val, Leu, He; (2) neutral hydrophilic: Cys, Ser, Thr, Asn, Gin; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class.

Particular conservative substitutions include, for example; Ala into Gly or into Ser; Arg into Lys; Asn into Gin or into H is; Asp into Glu; Cys into Ser; Gin into Asn; Glu into Asp; Gly into Ala or into Pro; His into Asn or into Gin; lie into Leu or into Val; Leu into lie or into Val; Lys into Arg, into Gin or into Glu; Met into Leu, into Tyr or into lie; Phe into Met, into Leu or into Tyr; Ser into Thr; Thr into Ser; Trp into Tyr; Tyr into Trp; and/or Phe into Val, into lie or into Leu.

In some embodiments, the antibody and/or antigen-binding portion thereof described herein can be a variant of a sequence described herein, e.g. a conservative substitution variant of an antibody polypeptide. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity, e.g. antigen-specific binding activity for the relevant target polypeptide, e.g. a sMIC polypeptide. A wide variety of PCR-based site-specific mutagenesis approaches are also known in the art and can be applied by the ordinarily skilled artisan.

In some embodiments, the antibodies described herein are not naturally-occurring biomolecules. For example, a murine antibody raised against an antigen of human origin would not occur in nature absent human intervention and manipulation, e.g. manufacturing steps carried out by a human. Chimeric antibodies are also not naturally-occurring biomolecules, e.g., in that they comprise sequences obtained from multiple species and assembled into a recombinant molecule. In certain particular embodiments, the human antibody reagents described herein are not naturally-occurring biomolecules, e.g., fully human antibodies directed against a human antigen would be subject to negative selection in nature and are not naturally found in the human body.

### III. IMMUNE CHECKPOINT INHIBITOR

In some embodiments, the present disclosure provides methods of combined targeting of sMIC and immune checkpoints. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular aspects, the immune checkpoint inhibitors target the PD-1 axis and/or CTLA-4.

The immune checkpoint inhibitors may be drugs such as small molecules, recombinant forms of ligand or receptors (e.g., soluble PD1), or, in particular, are antibodies, such as human antibodies (e.g., International Patent Publication WO 2015/016718; Pardoll, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example it is known that lambrolizumab is also known under the alternative and equivalent names MK-3475 and pembrolizumab.

It is contemplated that any of the immune checkpoint inhibitors that are known in the art to stimulate immune responses may be used. This includes inhibitors that directly or indirectly stimulate or enhance antigen-specific T-lymphocytes. These immune checkpoint inhibitors include, without limitation, agents targeting immune checkpoint proteins and pathways involving PD-L2, LAG3, BTLA, B7H4 and TIM3. For example, LAG3 inhibitors known in the art include soluble LAG3 (IMP321, or LAG3-Ig disclosed in WO 2009/044273) as well as mouse or humanized antibodies blocking human LAG3 (e.g., IMP701 disclosed in WO 2008/132601), or fully human antibodies blocking human LAG3 (such as disclosed in EP 2320940). Another example is provided by the use of blocking agents towards BTLA, including without limitation antibodies blocking human BTLA interaction with its ligand (such as 4C7 disclosed in WO 2011/014438). Yet another example is provided by the use of agents neutralizing B7H4 including without limitation antibodies to human B7H4 (disclosed in WO 2013/025779, and in WO 2013/067492) or soluble recombinant forms of B7H4 (such as disclosed in US 2012/0177645). Yet another example is provided by agents neutralizing B7-H3, including without limitation antibodies neutralizing human B7-H3 (e.g. MGA271 disclosed as BRCA84D and derivatives in U.S. Pub. No. 2012/0294796). Yet another example is provided by agents targeting TIM3, including without limitation antibodies targeting human TIM3 (e.g. as disclosed in WO 2013/006490 A2 or the anti-human TIM3, blocking antibody F38-2E2 disclosed by Jones et al., 2008).

In addition, more than one immune checkpoint inhibitor (e.g., anti-PD-1 antibody and anti-CTLA-4 antibody) may be used in combination with targeting sMIC. For example, anti-sMIC antibody and immune checkpoint inhibitors (e.g., anti-CTLA-4 antibody and/or anti-PD-1 antibody) can be administered to treat cancer.

A. PD-1 Axis Antagonists

T cell dysfunction or anergy occurs concurrently with an induced and sustained expression of the inhibitory receptor, programmed death 1 polypeptide (PD-1). Thus, therapeutic targeting of PD-1 and other molecules which signal through interactions with PD-1, such as programmed death ligand 1 (PD-L1) and programmed death ligand 2 (PD-L2) is provided herein. PD-L1 is overexpressed in many cancers and is often associated with poor prognosis (Okazaki et al., 2007). Thus, inhibition of the PD-L1/PD-1 interaction in combination with targeting of sMIC is provided herein such as to enhance CD8⁺ T cell-mediated killing of tumors.

Provided herein is a method for treating or delaying progression of cancer in an individual comprising administering to the individual an effective amount of a PD-1 axis binding antagonist in combination with anti-sMIC antibodies or fragments thereof. Also provided herein is a method of enhancing immune function in an individual in need thereof comprising administering to the individual an effective amount of a PD-1 axis binding antagonist and an anti-sMIC antibody or fragment thereof.

For example, a PD-1 axis binding antagonist includes a PD-1 binding antagonist, a PDL1 binding antagonist and a PDL2 binding antagonist. Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesion, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. 2014/0294898, 2014/022021, and 2011/0008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO 2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO 2010/027827 and WO 2011/066342. Additional PD-1 binding antagonists include Pidilizumab, also known as CT-011, MEDI0680, also known as AMP-514, and REGN2810.

In some aspects, the immune checkpoint inhibitor is a PD-L1 antagonist such as Durvalumab, also known as MEDI4736, atezolizumab, also known as MPDL3280A, or avelumab, also known as MSB00010118C. In certain aspects, the immune checkpoint inhibitor is a PD-L2 antagonist such as rHIgM12B7. In some aspects, the immune checkpoint inhibitor is a LAG-3 antagonist such as, but not limited to, IMP321, and BMS-986016. The immune checkpoint inhibitor may be an adenosine A2a receptor (A2aR) antagonist such as PBF-509.

In some aspects, the antibody described herein (such as an anti-PD-1 antibody, an anti-PDL1 antibody, or an anti-PDL2 antibody) further comprises a human or murine constant region. In a still further aspect, the human constant region is selected from the group consisting of IgG1, IgG2, IgG2, IgG3, IgG4. In a still further specific aspect, the human constant region is IgG1. In a still further aspect, the murine constant region is selected from the group consisting of IgG1, IgG2A, IgG2B, IgG3. In a still further specific aspect, the antibody has reduced or minimal effector function. In a still further specific aspect, the minimal effector function results from production in prokaryotic cells. In a still further specific aspect the minimal effector function results from an “effector-less Fc mutation” or aglycosylation.

Accordingly, an antibody used herein can be aglycosylated. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxy amino acid, most commonly serine or threonine, although 5-hydroxyproline or 5-hydroxy lysine may also be used. Removal of glycosylation sites form an antibody is conveniently accomplished by altering the amino acid sequence such that one of the above-described tripeptide sequences (for N-linked glycosylation sites) is removed. The alteration may be made by substitution of an asparagine, serine or threonine residue within the glycosylation site another amino acid residue (e.g., glycine, alanine or a conservative substitution).

The antibody or antigen binding fragment thereof, may be made using methods known in the art, for example, by a process comprising culturing a host cell containing nucleic acid encoding any of the previously described anti-PDL1, anti-PD-1, or anti-PDL2 antibodies or antigen-binding fragment in a form suitable for expression, under conditions suitable to produce such antibody or fragment, and recovering the antibody or fragment.

B. CTLA-4

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/014424, WO 98/042752; WO 00/037504 (CP675,206, also known as tremelimumab; formerly ticilimumab), U.S. Pat. No. 6,207,156; Hurwitz et al., 1998; Camacho et al., 2004; Mokyr et al., 1998, can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO 2001/014424, WO 2000/037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/014424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO 1995/001994 and WO 1998/042752; all incorporated herein by reference, and immunoadhesions such as described in U.S. Pat. No. 8,329,867, incorporated herein by reference.

C. Killer Immunoglobulin-Like Receptor (KIR)

Another immune checkpoint inhibitor for use in the present disclosure is an anti-MR antibody. Anti-human-MR antibodies (or VH/VL domains derived therefrom) suitable for use in the present methods can be generated using methods well known in the art.

Alternatively, art recognized anti-KIR antibodies can be used. The anti-MR antibody can be cross-reactive with multiple inhibitory MR receptors and potentiates the cytotoxicity of NK cells bearing one or more of these receptors. For example, the anti-MR antibody may bind to each of KIR2D2DL1, KIR2DL2, and KIR2DL3, and potentiate NK cell activity by reducing, neutralizing and/or reversing inhibition of NK cell cytotoxicity mediated by any or all of these KIRs. In some aspects, the anti-MR antibody does not bind KIR2DS4 and/or KIR2DS3. For example, monoclonal antibodies 1-7F9 (also known as IPH2101), 14F1, 1-6F1 and 1-6F5, described in WO 2006/003179, the teachings of which are hereby incorporated by reference, can be used. Antibodies that compete with any of these art-recognized antibodies for binding to MR also can be used. Additional art-recognized anti-MR antibodies which can be used include, for example, those disclosed in WO 2005/003168, WO 2005/009465, WO 2006/072625, WO 2006/072626, WO 2007/042573, WO 2008/084106, WO 2010/065939, WO 2012/071411 and WO/2012/160448.

An exemplary anti-MR antibody is lirilumab (also referred to as BMS-986015 or IPH2102). In other embodiments, the anti-MR antibody comprises the heavy and light chain complementarity determining regions (CDRs) or variable regions (VRs) of lirilumab. Accordingly, in one embodiment, the antibody comprises the CDR1, CDR2, and CDR3 domains of the heavy chain variable (VH) region of lirilumab, and the CDR1, CDR2 and CDR3 domains of the light chain variable (VL) region of lirilumab. In another embodiment, the antibody has at least about 90% variable region amino acid sequence identity with lirilumab.

D. Recombinant Oncolytic Viruses

Further embodiments of the present disclosure provide methods and compositions for cancer treatment comprising administering a recombinant oncolytic virus, such as a myxoma virus expressing a soluble form of PD1 (e.g., described in U.S. Provisional Patent Application No. 62/259,114; incorporated herein by reference), in combination with an anti-sMIC antibody. In these embodiments, the soluble PD1 protein binds to the PDL1 on local tumor cells thus preventing PDL1 from engaging the inhibitory PD1 expressed on tumor infiltration T cells.

In one aspect, the present disclosure generally pertains to recombinant, replication competent, oncolytic viruses. In one embodiment, there is provided a recombinant oncolytic virus having a heterologous nucleic acid sequence encoding PD-1. Oncolytic viruses that can be administered according to the methods of the invention include, without limitation, adenoviruses (e.g. Delta-24, Delta-24-RGD, ICOVIR-5, ICOVIR-7, Onyx-015, ColoAd1, H101, AD5/3-D24-GMCSF), reoviruses, herpes simplex virus (HSV; OncoVEX GMCSF), Newcastle Disease virus, measles viruses, retroviruses (e.g. influenza viruses), poxviruses (e.g. vaccinia virus including Copenhagen, Western Reserve, Wyeth strains), myxoma viruses, rhabdoviruses (e.g. vesicular stomatitis virus (VSV)), picornaviruses (e.g. Seneca Valley virus; SVV-001), coxsackievirus and parvovirus.

The recombinant virus can be constructed by procedures known in the art to generate recombinant viruses. An expression cassette encoding PD1 is inserted into the genome of an oncolytic virus at a region nonessential for viral replication. For example, the expression cassette is integrated in myxoma virus at an intergenic region, such as between the M135 and M136 open reading frames. The recombinant virus can comprise an expression cassette encoding a nucleotide sequence which is at least about 50%, 55%, 60%, 65%, 70%, 75%, 80%, 85%, 90%, 91%, 92%, 93%, 94%, 95%, 96%, 97%, 98%, 99% or more identical to the nucleotide sequence (e.g., to the entire length of the nucleotide sequence) of the extracellular portion of human PD1.

Homologous recombination (HR), also known as general recombination, is a type of genetic recombination used in all forms of life in which nucleotide sequences are exchanged between two similar or identical strands of DNA. The technique has been the standard method for genome engineering in mammalian cells since the mid-1980s. The process involves several steps of physical breaking and the eventual rejoining of DNA. This process is most widely used to repair potentially lethal double-strand breaks in DNA. In addition, homologous recombination produces new combinations of DNA sequences during meiosis, the process by which eukaryotes make germ cells like sperm and ova. These new combinations of DNA represent genetic variation in offspring which allow populations to evolutionarily adapt to changing environmental conditions over time. Homologous recombination is also used in horizontal gene transfer to exchange genetic material between different strains and species of bacteria and viruses. Homologous recombination is also used as a technique in molecular biology for introducing genetic changes into target organisms.

Expression cassettes included in vectors useful in the invention preferably contain (in a 5′-to-3′ direction) a eukaryotic transcriptional promoter operably linked to a protein-coding sequence. Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter (Ng, 1989; Quitsche et al., 1989), GADPH promoter (Alexander et al., 1988, Ercolani et al., 1988), metallothionein promoter (Karin et al., 1989; Richards et al., 1984); and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). A specific example could be a synthetic early/late (sE/L) poxvirus promoter.

The expression cassette is introduced to cells which are then infected with the unmodified oncolytic virus to produce the recombinant virus. Introduction of the expression cassette into cells may use any suitable methods for nucleic acid delivery for transformation of a cell, as described herein or as would be known to one of ordinary skill in the art. Such methods include, but are not limited to, direct delivery of DNA such as by ex vivo transfection (Wilson et al., 1989, Nabel et al, 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

The recombinant virus is then purified from the cells such as by a selectable marker. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker. Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. The marker used is not believed to be important, so long as it is capable of being expressed simultaneously with the nucleic acid encoding a gene product. For example, the recombinant oncolytic virus can be untagged or express fluorescent proteins such as green fluorescent protein (GFP), red fluorescent protein (RFP), tomato Red (tdRed), or other fluorescent proteins. Further examples of selection and screenable markers are well known to one of skill in the art.

### IV. METHODS OF USE

Further provided herein are methods for treating or delaying progression of cancer in an individual comprising administering to the individual an effective amount of at least one immune checkpoint inhibitor and an anti-sMIC antibody or antigen-binding fragment thereof.

Examples of cancer include but are not limited to, carcinoma, lymphoma, blastoma, sarcoma, and leukemia. More particular examples of such cancers include, but are not limited to, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

Also provided herein are methods of determining a response to an immune checkpoint inhibitor in a patient having a cancer by measuring the level of serum sMIC in a sample obtained from the subject, particularly a blood sample. A patient determined to have a low or absent serum sMIC level is predicted to have a favorable response to immune checkpoint inhibitors. On the other hand, a subject determined to have a high serum sMIC level is predicted to have an adverse reaction to an immune checkpoint inhibitor. Accordingly, methods are provided for treating a subject with a high serum sMIC level by administering an anti-sMIC antibody in combination with the immune checkpoint inhibitor.

In some embodiments, the tumor or malignancy is MIC-positive. As used herein, the term “MIC-positive tumor” is used to describe a tumor cell, a cluster of tumor cells or a tumor mass, which produces a MIC protein. This term is intended to encompass all tumor cells and/or tumor masses that shed all or part of a MIC protein, thus these cells may only display a MIC protein on its surface for a short time period—that is, the term encompasses tumors that shed MIC protein, regardless of whether detectable MIC protein remains present on their cell surface or not. However, any tumor that is capable of escaping innate immune rejection by shedding MIC is considered to be a “MIC-positive tumor” as that term is used herein. Some non-limiting examples of MIC-positive tumors include epithelial cell tumors and hematopoietic malignancies. In some embodiments, the MIC-positive tumor or malignancy can be a MIC-positive prostate cancer and/or metastasis thereof.

Further embodiments provide methods for the treatment of colitis in a subject. In one method, the subject is administered an effective amount of an anti-sMIC antibody. The colitis treatable in the present disclosure includes infectious colitis and inflammatory bowel diseases. The inflammatory bowel diseases include but are not limited to idiopathic, chronic, and non-specific colitis such as ulcerative colitis and Crohn's disease, as well as typical inflammatory conditions of the colon and small intestine. The colitis may be infectious colitis, such as amoebic dysentery, giardiasis, intestinal tuberculosis, mycosis, or non-specific colitis such as Crohn's disease, or ulcerative colitis. Crohn's disease is a type of inflammatory bowel disease that may affect any part of the gastrointestinal tract from the mouth to the anus. In some aspects, a subject having colitis and being treated according to the embodiments may have one or more active fistula. The antibody may be administered in combination with a second colitis therapeutic, such as anti-inflammatory, immunosuppressant, antibiotic, pain reliever, and/or surgery. For example, the anti-sMIC antibody may be combined with aminosalicylates such as sulfasalazine, mesalazine, balsalazide, and olsalazine; corticosteroid such as prednisone and hydrocortisone; and/or immunosuppressants such as azathioprine, mercaptopurine, cyclosporingm infliximab, adalimumab, golimumab, and vedolizumab.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various cancers. In addition, the methods described herein can be used to treat domesticated animals and/or pets. A subject can be male or female.

A. Administration

The combination therapy provided herein comprises administration of an immune checkpoint inhibitor (e.g., PD-1 axis binding antagonist and/or CTLA-4 antibody) and an anti-sMIC antibody or antigen-binding fragment thereof. The combination therapy may be administered in any suitable manner known in the art. For example, of an immune checkpoint inhibitor (e.g., PD-1 axis binding antagonist and/or CTLA-4 antibody) and an anti-sMIC antibody or antigen-binding fragment thereof may be administered sequentially (at different times) or concurrently (at the same time). In some embodiments, the one or more immune checkpoint inhibitors are in a separate composition as the anti-sMIC antibody or antigen-binding fragment thereof. In some embodiments, the immune checkpoint inhibitor is in the same composition as the anti-sMIC antibody or antigen-binding fragment thereof. In certain aspects, the subject is administered the anti-sMIC antibody or antigen-binding fragment thereof before, simultaneously, or after the at least one immune checkpoint inhibitor.

The one or more immune checkpoint inhibitors and the anti-sMIC antibody or antigen-binding fragment thereof may be administered by the same route of administration or by different routes of administration. In some embodiments, the immune checkpoint inhibitor is administered intravenously, intramuscularly, subcutaneously, topically, orally, transdermally, intraperitoneally, intraorbitally, by implantation, by inhalation, intrathecally, intraventricularly, or intranasally. In some embodiments, the anti-sMIC antibody or antigen-binding fragment thereof is administered intravenously, intramuscularly, subcutaneously, topically, orally, transdermally, intraperitoneally, intraorbitally, by implantation, by inhalation, intrathecally, intraventricularly, or intranasally. An effective amount of the immune checkpoint inhibitor and the anti-sMIC antibody or antigen-binding fragment thereof may be administered for prevention or treatment of disease. The appropriate dosage of immune checkpoint inhibitor and/or the anti-sMIC antibody or antigen-binding fragment thereof may be determined based on the type of disease to be treated, severity and course of the disease, the clinical condition of the individual, the individual's clinical history and response to the treatment, and the discretion of the attending physician. In some embodiments, combination treatment with at least one immune checkpoint inhibitor (e.g., PD-1 axis binding antagonist and/or CTLA-4 antibody) and anti-sMIC antibody or antigen-binding fragment thereof are synergistic, whereby an efficacious dose of an anti-sMIC antibody or antigen-binding fragment thereof in the combination is reduced relative to efficacious dose of at the least one immune checkpoint inhibitor (e.g., PD-1 axis binding antagonist and/or CTLA-4 antibody) as a single agent.

The dosage ranges for the agent depend upon the potency, and encompass amounts large enough to produce the desired effect e.g., slowing of tumor growth or a reduction in tumor size. The dosage should not be so large as to cause unacceptable adverse side effects. Generally, the dosage will vary with the age, condition, and sex of the patient and can be determined by one of skill in the art. The dosage can also be adjusted by the individual physician in the event of any complication. In some embodiments, the dosage ranges from 0.001 mg/kg body weight to 0.5 mg/kg body weight. In some embodiments, the dose range is from 5 μg/kg body weight to 100 μg/kg body weight. Alternatively, the dose range can be titrated to maintain serum levels between 1 μg/mL and 1000 μg/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

Administration of the doses recited above can be repeated. In fact, to the extent that inhibition of MIC shedding can promote immune attack on a tumor, long term administration is contemplated, e.g. first to treat the tumor itself, and then to provide continued surveillance against the development of tumor cells that gain the ability to shed MIC. In some embodiments, the doses are given once a day, or multiple times a day, for example but not limited to three times a day. In a preferred embodiment, the doses recited above are administered daily for several weeks or months. The duration of treatment depends upon the subject's clinical progress and responsiveness to therapy.

In some embodiments, the dose can be from about 2 mg/kg to about 15 mg/kg. In some embodiments, the dose can be about 2 mg/kg. In some embodiments, the dose can be about 4 mg/kg. In some embodiments, the dose can be about 5 mg/kg. In some embodiments, the dose can be about 6 mg/kg. In some embodiments, the dose can be about 8 mg/kg. In some embodiments, the dose can be about 10 mg/kg. In some embodiments, the dose can be about 15 mg/kg. In some embodiments, the dose can be from about 100 mg/m² to about 700 mg/m². In some embodiments, the dose can be about 250 mg/m². In some embodiments, the dose can be about 375 mg/m². In some embodiments, the dose can be about 400 mg/m². In some embodiments, the dose can be about 500 mg/m².

In some embodiments, the dose can be administered intravenously. In some embodiments, the intravenous administration can be an infusion occurring over a period of from about 10 minute to about 3 hours. In some embodiments, the intravenous administration can be an infusion occurring over a period of from about 30 minutes to about 90 minutes.

In some embodiments the dose can be administered about weekly. In some embodiments, the dose can be administered weekly. In some embodiments, the dose can be administered weekly for from about 12 weeks to about 18 weeks. In some embodiments the dose can be administered about every 2 weeks. In some embodiments the dose can be administered about every 3 weeks. In some embodiments, the dose can be from about 2 mg/kg to about 15 mg/kg administered about every 2 weeks. In some embodiments, the dose can be from about 2 mg/kg to about 15 mg/kg administered about every 3 weeks. In some embodiments, the dose can be from about 2 mg/kg to about 15 mg/kg administered intravenously about every 2 weeks. In some embodiments, the dose can be from about 2 mg/kg to about 15 mg/kg administered intravenously about every 3 weeks. In some embodiments, the dose can be from about 200 mg/m² to about 400 mg/m² administered intravenously about every week. In some embodiments, the dose can be from about 200 mg/m² to about 400 mg/m² administered intravenously about every 2 weeks. In some embodiments, the dose can be from about 200 mg/m² to about 400 mg/m² administered intravenously about every 3 weeks. In some embodiments, a total of from about 2 to about 10 doses are administered. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, the administration occurs for a total of from about 4 weeks to about 12 weeks. In some embodiments, the administration occurs for a total of about 6 weeks. In some embodiments, the administration occurs for a total of about 8 weeks. In some embodiments, the administration occurs for a total of about 12 weeks. In some embodiments, the initial dose can be from about 1.5 to about 2.5 fold greater than subsequent doses.

Oncolytic viruses according to the invention may be administered in a single administration or multiple administrations. The virus may be administered at dosage of 1×10⁵ plaque forming units (PFU), 5×10⁵ PFU, at least 1×10⁶ PFU, 5×10⁶ or about 5×10⁶ PFU, 1×10⁷, at least 1×10⁷ PFU, 1×10⁸ or about 1×10⁸ PFU, at least 1×10⁸ PFU, about or at least 5×10⁸ PFU, 1×10⁹ or at least 1×10⁹ PFU, 5×10⁹ or at least 5×10⁹ PFU, 1×10¹⁰ PFU or at least 1×10¹⁰ PFU, 5×10¹⁰ or at least 5×10¹⁰ PFU, 1×10¹¹ or at least 1×10¹¹, 1×10¹² or at least 1×10¹², 1×10¹³ or at least 1×10¹³. For example, the virus may be administered at a dosage of between about 10⁷-10¹³, between about 10⁸-10¹³, between about 10⁹-10¹², or between about 10⁸-10¹².

B. Additional Therapies

In order to increase the effectiveness of the anti-sMIC antibody or antigen-binding fragment thereof and the at least one immune checkpoint inhibitor, they can be combined with at least one additional agent effective in the treatment of cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s). Alternatively, the expression construct may contact the proliferating cell and the additional therapy may affect other cells of the immune system or the tumor microenvironment to enhance anti-tumor immune responses and therapeutic efficacy. The at least one additional anticancer therapy may be, without limitation, a surgical therapy, chemotherapy (e.g., administration of a protein kinase inhibitor or a EGFR-targeted therapy), radiation therapy, cryotherapy, hyperthermia treatment, phototherapy, radioablation therapy, hormonal therapy, immunotherapy, small molecule therapy, receptor kinase inhibitor therapy, anti-angiogenic therapy, cytokine therapy or a biological therapies such as monoclonal antibodies, siRNA, miRNA, antisense oligonucleotides, ribozymes or gene therapy. Without limitation the biological therapy may be a gene therapy, such as tumor suppressor gene therapy, a cell death protein gene therapy, a cell cycle regulator gene therapy, a cytokine gene therapy, a toxin gene therapy, an immunogene therapy, a suicide gene therapy, a prodrug gene therapy, an anti-cellular proliferation gene therapy, an enzyme gene therapy, or an anti-angiogenic factor gene therapy.

The combination therapy may precede or follow the other agent treatment by intervals ranging from minutes to weeks. In embodiments where the other agent and combination therapy are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 hours of each other and, more preferably, within about 6-12 hours of each other. In some situations, it may be desirable to extend the time period for treatment significantly, however, where several days (e.g., 2, 3, 4, 5, 6 or 7) to several weeks (e.g., 1, 2, 3, 4, 5, 6, 7 or 8) lapse between the respective administrations.

Various combinations may be employed, an anti-sMIC antibody or antigen-binding fragment thereof and immune checkpoint inhibitor is “A” and the secondary agent, i.e. chemotherapy, is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

1. Chemotherapy

Cancer therapies in general also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy.

Yet further combination chemotherapies include, for example, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegaIl; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; carboplatin, procarbazine, plicomycin, gemcitabien, navelbine, farnesyl-protein transferase inhibitors, transplatinum; and pharmaceutically acceptable salts, acids or derivatives of any of the above. In certain embodiments, the compositions provided herein may be used in combination with histone deacetylase inhibitors. In certain embodiments, the compositions provided herein may be used in combination with gefitinib. In other embodiments, the present embodiments may be practiced in combination with Gleevec (e.g., from about 400 to about 800 mg/day of Gleevec may be administered to a patient). In certain embodiments, one or more chemotherapeutic may be used in combination with the compositions provided herein.

2. Radiotherapy

Other factors that cause DNA damage and have been used extensively include what are commonly known as y-rays, X-rays, and/or the directed delivery of radioisotopes to tumor cells. Other forms of DNA damaging factors are also known such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells as well as genetically engineered variants of these cell types modified to express chimeric antigen receptors.

Examples of immunotherapies that can be combined with the anti-sMIC antibody or antigen-binding fragment thereof and immune checkpoint inhibitor are immune adjuvants (e.g., Mycobacterium bovis, Plasmodium falciparum, dinitrochlorobenzene and aromatic compounds) (U.S. Pat. Nos. 5,801,005; 5,739,169; Hui and Hashimoto, 1998; Christodoulides et al., 1998), cytokine therapy (e.g., interferons α, β and γ; interleukins (IL-1, IL-2), GM-CSF and TNF) (Bukowski et al., 1998; Davidson et al., 1998; Hellstrand et al., 1998) gene therapy (e.g., TNF, IL-1, IL-2, p53) (Qin et al., 1998; Austin-Ward and Villaseca, 1998; U.S. Pat. Nos. 5,830,880 and 5,846,945) and monoclonal antibodies (e.g., anti-ganglioside GM2, anti-HER-2, anti-p185) (Pietras et al., 1998; Hanibuchi et al., 1998; U.S. Pat. No. 5,824,311). Herceptin (trastuzumab) is a chimeric (mouse-human) monoclonal antibody that blocks the HER2-neu receptor. It possesses anti-tumor activity and has been approved for use in the treatment of malignant tumors (Dillman, 1999). Combination therapy of cancer with herceptin and chemotherapy has been shown to be more effective than the individual therapies. Thus, it is contemplated that one or more anti-cancer therapies may be employed with the combination therapy described herein.

Other immunotherapies contemplated for use in methods of the present disclosure include those described by Tchekmedyian et al., 2015, incorporated herein by reference. The immunotherapy may comprise suppression of T regulatory cells (Tregs), myeloid derived suppressor cells (MDSCs) and cancer associated fibroblasts (CAFs). In some embodiments, the immunotherapy is a tumor vaccine (e.g., whole tumor cell vaccines, peptides, and recombinant tumor associated antigen vaccines), or adoptive cellular therapies (ACT) (e.g., T cells, natural killer cells, TILs, and LAK cells). The T cells may be engineered with chimeric antigen receptors (CARs) or T cell receptors (TCRs) to specific tumor antigens. As used herein, a chimeric antigen receptor (or CAR) may refer to any engineered receptor specific for an antigen of interest that, when expressed in a T cell, confers the specificity of the CAR onto the T cell. Once created using standard molecular techniques, a T cell expressing a chimeric antigen receptor may be introduced into a patient, as with a technique such as adoptive cell transfer. In some aspects, the T cells are activated CD4 and/or CD8 T cells in the individual which are characterized by γ-IFN “producing CD4 and/or CD8 T cells and/or enhanced cytolytic activity relative to prior to the administration of the combination. The CD4 and/or CD8 T cells may exhibit increased release of cytokines selected from the group consisting of IFN-γ, TNF-α and interleukins. The CD4 and/or CD8 T cells can be effector memory T cells. In certain embodiments, the CD4 and/or CD8 effector memory T cells are characterized by having the expression of CD44^{high} CD62^{low}.

The immunotherapy may be a cancer vaccine comprising one or more cancer antigens, in particular a protein or an immunogenic fragment thereof, DNA or RNA encoding said cancer antigen, in particular a protein or an immunogenic fragment thereof, cancer cell lysates, and/or protein preparations from tumor cells. As used herein, a cancer antigen is an antigenic substance present in cancer cells. In principle, any protein produced in a cancer cell that has an abnormal structure due to mutation can act as a cancer antigen. In principle, cancer antigens can be products of mutated Oncogenes and tumor suppressor genes, products of other mutated genes, overexpressed or aberrantly expressed cellular proteins, cancer antigens produced by oncogenic viruses, oncofetal antigens, altered cell surface glycolipids and glycoproteins, or cell type-specific differentiation antigens. Examples of cancer antigens include the abnormal products of ras and p53 genes. Other examples include tissue differentiation antigens, mutant protein antigens, oncogenic viral antigens, cancer-testis antigens and vascular or stromal specific antigens. Tissue differentiation antigens are those that are specific to a certain type of tissue. Mutant protein antigens are likely to be much more specific to cancer cells because normal cells shouldn't contain these proteins. Normal cells will display the normal protein antigen on their MHC molecules, whereas cancer cells will display the mutant version. Some viral proteins are implicated in forming cancer, and some viral antigens are also cancer antigens. Cancer-testis antigens are antigens expressed primarily in the germ cells of the testes, but also in fetal ovaries and the trophoblast. Some cancer cells aberrantly express these proteins and therefore present these antigens, allowing attack by T-cells specific to these antigens. Exemplary antigens of this type are CTAG1 B and MAGEA1 as well as Rindopepimut, a 14-mer intradermal injectable peptide vaccine targeted against epidermal growth factor receptor (EGFR) v111 variant. Rindopepimut is particularly suitable for treating glioblastoma when used in combination with an inhibitor of the CD95/CD95L signaling system as described herein. Also, proteins that are normally produced in very low quantities, but whose production is dramatically increased in cancer cells, may trigger an immune response. An example of such a protein is the enzyme tyrosinase, which is required for melanin production. Normally tyrosinase is produced in minute quantities but its levels are very much elevated in melanoma cells. Oncofetal antigens are another important class of cancer antigens. Examples are alphafetoprotein (AFP) and carcinoembryonic antigen (CEA). These proteins are normally produced in the early stages of embryonic development and disappear by the time the immune system is fully developed. Thus self-tolerance does not develop against these antigens. Abnormal proteins are also produced by cells infected with oncoviruses, e.g. EBV and HPV. Cells infected by these viruses contain latent viral DNA which is transcribed and the resulting protein produces an immune response. A cancer vaccine may include a peptide cancer vaccine, which in some embodiments is a personalized peptide vaccine. In some embodiments, the peptide cancer vaccine is a multivalent long peptide vaccine, a multi-peptide vaccine, a peptide cocktail vaccine, a hybrid peptide vaccine, or a peptide-pulsed dendritic cell vaccine

The immunotherapy may be an antibody, such as part of a polyclonal antibody preparation, or may be a monoclonal antibody. The antibody may be a humanized antibody, a chimeric antibody, an antibody fragment, a bispecific antibody or a single chain antibody. An antibody as disclosed herein includes an antibody fragment, such as, but not limited to, Fab, Fab′ and F(ab′)2, Fd, single-chain Fvs (scFv), single-chain antibodies, disulfide-linked Fvs (sdfv) and fragments including either a VL or VH domain. In some aspects, the antibody or fragment thereof specifically binds epidermal growth factor receptor (EGFR1, Erb-B1), HER2/neu (Erb-B2), CD20, Vascular endothelial growth factor (VEGF), insulin-like growth factor receptor (IGF-1R), TRAIL-receptor, epithelial cell adhesion molecule, carcino-embryonic antigen, Prostate-specific membrane antigen, Mucin-1, CD30, CD33, or CD40.

Examples of monoclonal antibodies that may be used in combination with the compositions provided herein include, without limitation, trastuzumab (anti-HER2/neu antibody); Pertuzumab (anti-HER2 mAb); cetuximab (chimeric monoclonal antibody to epidermal growth factor receptor EGFR); panitumumab (anti-EGFR antibody); nimotuzumab (anti-EGFR antibody); Zalutumumab (anti-EGFR mAb); Necitumumab (anti-EGFR mAb); MDX-210 (humanized anti-HER-2 bispecific antibody); MDX-210 (humanized anti-HER-2 bispecific antibody); MDX-447 (humanized anti-EGF receptor bispecific antibody); Rituximab (chimeric murine/human anti-CD20 mAb); Obinutuzumab (anti-CD20 mAb); Ofatumumab (anti-CD20 mAb); Tositumumab-I131 (anti-CD20 mAb); Ibritumomab tiuxetan (anti-CD20 mAb); Bevacizumab (anti-VEGF mAb); Ramucirumab (anti-VEGFR2 mAb); Ranibizumab (anti-VEGF mAb); Aflibercept (extracellular domains of VEGFR1 and VEGFR2 fused to IgG1 Fc); AMG386 (angiopoietin-1 and -2 binding peptide fused to IgG1 Fc); Dalotuzumab (anti-IGF-1R mAb); Gemtuzumab ozogamicin (anti-CD33 mAb); Alemtuzumab (anti-Campath-1/CD52 mAb); Brentuximab vedotin (anti-CD30 mAb); Catumaxomab (bispecific mAb that targets epithelial cell adhesion molecule and CD3); Naptumomab (anti-5T4 mAb); Girentuximab (anti-Carbonic anhydrase ix); or Farletuzumab (anti-folate receptor). Other examples include antibodies such as Panorex™ (17-1A) (murine monoclonal antibody); Panorex (@ (17-1A) (chimeric murine monoclonal antibody); BEC2 (ami-idiotypic mAb, mimics the GD epitope) (with BCG); Oncolym (Lym-1 monoclonal antibody); SMART M195 Ab, humanized 13′ 1 LYM-1 (Oncolym), Ovarex (B43.13, anti-idiotypic mouse mAb); 3622W94 mAb that binds to EGP40 (17-1A) pancarcinoma antigen on adenocarcinomas; Zenapax (SMART Anti-Tac (IL-2 receptor); SMART M195 Ab, humanized Ab, humanized); NovoMAb-G2 (pancarcinoma specific Ab); TNT (chimeric mAb to histone antigens); TNT (chimeric mAb to histone antigens); Gliomab-H (Monoclonals-Humanized Abs); GNI-250 Mab; EMD-72000 (chimeric-EGF antagonist); LymphoCide (humanized IL.L.2 antibody); and MDX-260 bispecific, targets GD-2, ANA Ab, SMART IDIO Ab, SMART ABL 364 Ab or ImmuRAIT-CEA. Examples of antibodies include those disclosed in U.S. Pat. Nos. 5,736,167, 7,060,808, and 5,821,337.

Further examples of antibodies include Zanulimumab (anti-CD4 mAb), Keliximab (anti-CD4 mAb); Ipilimumab (MDX-101; anti-CTLA-4 mAb); Tremilimumab (anti-CTLA-4 mAb); (Daclizumab (anti-CD25/IL-2R mAb); Basiliximab (anti-CD25/IL-2R mAb); MDX-1106 (anti-PD1 mAb); antibody to GITR; GC1008 (anti-TGF-β antibody); metelimumab/CAT-192 (anti-TGF-β antibody); lerdelimumab/CAT-152 (anti-TGF-β antibody); ID11 (anti-TGF-β antibody); Denosumab (anti-RANKL mAb); BMS-663513 (humanized anti-4-1BB mAb); SGN-40 (humanized anti-CD40 mAb); CP870,893 (human anti-CD40 mAb); Infliximab (chimeric anti-TNF mAb; Adalimumab (human anti-TNF mAb); Certolizumab (humanized Fab anti-TNF); Golimumab (anti-TNF); Etanercept (Extracellular domain of TNFR fused to IgG1 Fc); Belatacept (Extracellular domain of CTLA-4 fused to Fc); Abatacept (Extracellular domain of CTLA-4 fused to Fc); Belimumab (anti-B Lymphocyte stimulator); Muromonab-CD3 (anti-CD3 mAb); Otelixizumab (anti-CD3 mAb); Teplizumab (anti-CD3 mAb); Tocilizumab (anti-IL6R mAb); REGN88 (anti-IL6R mAb); Ustekinumab (anti-IL-12/23 mAb); Briakinumab (anti-IL-12/23 mAb); Natalizumab (anti-α4 integrin); Vedolizumab (anti-α4 β7 integrin mAb); T1 h (anti-CD6 mAb); Epratuzumab (anti-CD22 mAb); Efalizumab (anti-CD11a mAb); and Atacicept (extracellular domain of transmembrane activator and calcium-modulating ligand interactor fused with Fc).

a. Passive Immunotherapy

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow.

Preferably, human monoclonal antibodies are employed in passive immunotherapy, as they produce few or no side effects in the patient. Human monoclonal antibodies to ganglioside antigens have been administered intralesionally to patients suffering from cutaneous recurrent melanoma (Irie & Morton, 1986). Regression was observed in six out of ten patients, following, daily or weekly, intralesional injections. In another study, moderate success was achieved from intralesional injections of two human monoclonal antibodies (Irie et al., 1989).

It may be favorable to administer more than one monoclonal antibody directed against two different antigens or even antibodies with multiple antigen specificity. Treatment protocols also may include administration of lymphokines or other immune enhancers as described by Bajorin et al., 1988. The development of human monoclonal antibodies is described in further detail elsewhere in the specification.

b. Active Immunotherapy

In active immunotherapy, an antigenic peptide, polypeptide or protein, or an autologous or allogenic tumor cell composition or “vaccine” is administered, generally with a distinct bacterial adjuvant (Ravindranath & Morton, 1991; Morton & Ravindranath, 1996; Morton et al., 1992; Mitchell et al., 1990; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

c. Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated antigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma, but the percentage of responders were few compared to those who did not respond. More recently, higher response rates have been observed when such adoptive immune cellular therapies have incorporated genetically engineered T cells that express chimeric antigen receptors (CAR) termed CAR T cell therapy. Similarly, natural killer cells both autologous and allogenic have been isolated, expanded and genetically modified to express receptors or ligands to facilitate their binding and killing of tumor cells.

4. Other Agents

It is contemplated that other agents may be used in combination with the compositions provided herein to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the compositions provided herein by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the compositions provided herein to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the compositions provided herein to improve the treatment efficacy.

In further embodiments, the other agents may be one or more oncolytic viruses. Examples of oncolytic viruses include adenoviruses, adeno-associated viruses, retroviruses, lentiviruses, herpes viruses, pox viruses, vaccinia viruses, vesicular stomatitis viruses, polio viruses, Newcastle's Disease viruses, Epstein-Barr viruses, influenza viruses and reoviruses. In a particular embodiment, the other agent is talimogene laherparepvec (T-VEC) which is an oncolytic herpes simplex virus genetically engineered to express GM-CSF. Talimogene laherparepvec, HSV-1 [strain JS1] ICP34.5-/ICP47-/hGM-CSF, (previously known as OncoVEX^{GM CSF}) is an intratumorally delivered oncolytic immunotherapy comprising an immune-enhanced HSV-1 that selectively replicates in solid tumors. (Lui et al., 2003; U.S. Pat. Nos. 7,223,593 and 7,537,924; incorporated herein by reference).

In certain embodiments, hormonal therapy may also be used in conjunction with the present embodiments or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases

In some aspects, the additional anti-cancer agent is a protein kinase inhibitor or a monoclonal antibody that inhibits receptors involved in protein kinase or growth factor signaling pathways such as an EGFR, VEGFR, AKT, Erb1, Erb2, ErbB, Syk, Bcr-Abl, JAK, Src, GSK-3, PI3K, Ras, Raf, MAPK, MAPKK, mTOR, c-Kit, eph receptor or BRAF inhibitors. Nonlimiting examples of protein kinase or growth factor signaling pathways inhibitors include Afatinib, Axitinib, Bevacizumab, Bosutinib, Cetuximab, Crizotinib, Dasatinib, Erlotinib, Fostamatinib, Gefitinib, Imatinib, Lapatinib, Lenvatinib, Mubritinib, Nilotinib, Panitumumab, Pazopanib, Pegaptanib, Ranibizumab, Ruxolitinib, Saracatinib, Sorafenib, Sunitinib, Trastuzumab, Vandetanib, AP23451, Vemurafenib, MK-2206, GSK690693, A-443654, VQD-002, Miltefosine, Perifosine, CAL101, PX-866, LY294002, rapamycin, temsirolimus, everolimus, ridaforolimus, Alvocidib, Genistein, Selumetinib, AZD-6244, Vatalanib, P1446A-05, AG-024322, ZD1839, P276-00, GW572016 or a mixture thereof.

In some aspects, the PI3K inhibitor is selected from the group of PI3K inhibitors consisting of buparlisib, idelalisib, BYL-719, dactolisib, PF-05212384, pictilisib, copanlisib, copanlisib dihydrochloride, ZSTK-474, GSK-2636771, duvelisib, GS-9820, PF-04691502, SAR-245408, SAR-245409, sonolisib, Archexin, GDC-0032, GDC-0980, apitolisib, pilaralisib, DLBS 1425, PX-866, voxtalisib, AZD-8186, BGT-226, DS-7423, GDC-0084, GSK-21 26458, INK-1 1 17, SAR-260301, SF-1 1 26, AMG-319, BAY-1082439, CH-51 32799, GSK-2269557, P-71 70, PWT-33597, CAL-263, RG-7603, LY-3023414, RP-5264, RV-1729, taselisib, TGR-1 202, GSK-418, INCB-040093, Panulisib, GSK-105961 5, CNX-1351, AMG-51 1, PQR-309, 17beta-Hydroxywortmannin, AEZS-129, AEZS-136, HM-5016699, IPI-443, ONC-201, PF-4989216, RP-6503, SF-2626, X-339, XL-499, PQR-401, AEZS-132, CZC-24832, KAR-4141, PQR-31 1, PQR-316, RP-5090, VS-5584, X-480, AEZS-126, AS-604850, BAG-956, CAL-130, CZC-24758, ETP-46321, ETP-471 87, GNE-317, GS-548202, HM-032, KAR-1 139, LY-294002, PF-04979064, PI-620, PKI-402, PWT-143, RP-6530, 3-HOI-BA-01, AEZS-134, AS-041 164, AS-252424, AS-605240, AS-605858, AS-606839, BCCA-621 C, CAY-10505, CH-5033855, CH-51 08134, CUDC-908, CZC-1 9945, D-106669, D-87503, DPT-NX7, ETP-46444, ETP-46992, GE-21, GNE-123, GNE-151, GNE-293, GNE-380, GNE-390, GNE-477, GNE-490, GNE-493, GNE-614, HMPL-51 8, HS-104, HS-1 06, HS-1 16, HS-173, HS-196, IC-486068, INK-055, KAR 1 141, KY-1 2420, Wortmannin, Lin-05, NPT-520-34, PF-04691503, PF-06465603, PGNX-01, PGNX-02, PI 620, PI-103, PI-509, PI-516, PI-540, PIK-75, PWT-458, RO-2492, RP-5152, RP-5237, SB-201 5, SB-2312, SB-2343, SHBM-1009, SN 32976, SR-13179, SRX-2523, SRX-2558, SRX-2626, SRX-3636, SRX-5000, TGR-5237, TGX-221, UCB-5857, WAY-266175, WAY-266176, EI-201, AEZS-131, AQX-MN100, KCC-TGX, OXY-1 1 1 A, PI-708, PX-2000, and WJD-008.

It is contemplated that the additional cancer therapy can comprise an antibody, peptide, polypeptide, small molecule inhibitor, siRNA, miRNA or gene therapy which targets, for example, epidermal growth factor receptor (EGFR, EGFR1, ErbB-1, HER1), ErbB-2 (HER2/neu), ErbB-3/HER3, ErbB-4/HER4, EGFR ligand family; insulin-like growth factor receptor (IGFR) family, IGF-binding proteins (IGFBPs), IGFR ligand family (IGF-1R); platelet derived growth factor receptor (PDGFR) family, PDGFR ligand family; fibroblast growth factor receptor (FGFR) family, FGFR ligand family, vascular endothelial growth factor receptor (VEGFR) family, VEGF family; HGF receptor family: TRK receptor family; ephrin (EPH) receptor family; AXL receptor family; leukocyte tyrosine kinase (LTK) receptor family; TIE receptor family, angiopoietin 1, 2; receptor tyrosine kinase-like orphan receptor (ROR) receptor family; discoidin domain receptor (DDR) family; RET receptor family; KLG receptor family; RYK receptor family; MuSK receptor family; Transforming growth factor alpha (TGF-α), TGF-α receptor; Transforming growth factor-beta (TGF-β), TGF-β receptor; Interleukin 13 receptor alpha2 chain (1L13Ralpha2), Interleukin-6 (IL-6), 1L-6 receptor, Interleukin-4, IL-4 receptor, Cytokine receptors, Class I (hematopoietin family) and Class II (interferon/1L-10 family) receptors, tumor necrosis factor (TNF) family, TNF-α, tumor necrosis factor (TNF) receptor superfamily (TNTRSF), death receptor family, TRAIL-receptor; cancer-testis (CT) antigens, lineage-specific antigens, differentiation antigens, alpha-actinin-4, ARTC1, breakpoint cluster region-Abelson (Bcr-abl) fusion products, B-RAF, caspase-5 (CASP-5), caspase-8 (CASP-8), beta-catenin (CTNNB1), cell division cycle 27 (CDC27), cyclin-dependent kinase 4 (CDK4), CDKN2A, COA-1, dek-can fusion protein, EFTUD-2, Elongation factor 2 (ELF2), Ets variant gene 6/acute myeloid leukemia 1 gene ETS (ETC6-AML1) fusion protein, fibronectin (FN), GPNMB, low density lipid receptor/GDP-L fucose: beta-Dgalactose 2-alpha-Lfucosyltraosferase (LDLR/FUT) fusion protein, HLA-A2, arginine to isoleucine exchange at residue 170 of the alpha-helix of the alpha2-domain in the HLA-A2 gene (HLA-A*201-R1700, MLA-A11, heat shock protein 70-2 mutated (HSP70-2M), KIAA0205, MART2, melanoma ubiquitous mutated 1, 2, 3 (MUM-1, 2, 3), prostatic acid phosphatase (PAP), neo-PAP, Myosin class 1, NFYC, OGT, OS-9, pml-RARalpha fusion protein, PRDXS, PTPRK, K-ras (KRAS2), N-ras (NRAS), HRAS, RBAF600, SIRT2, SNRPD1, SYT-SSX1 or -SSX2 fusion protein, Triosephosphate Isomerase, BAGE, BAGE-1, BAGE-2,3,4,5, GAGE-1,2,3,4,5,6,7,8, GnT-V (aberrant N-acetyl giucosaminyl transferase V, MGATS), HERV-K-MEL, KK-LC, LAGE, LAGE-1, CTL-recognixed antigen on melanoma (CAMEL), MAGE-A1 (MAGE-1), MAGE-A2, MAGE-A3, MAGE-A4, MAGE-AS, MAGE-A6, MAGE-A8, MAGE-A9, MAGE-A10, MAGE-A11, MAGE-A12, MAGE-3, MAGE-B1, MAGE-B2, MAGE-B5, MAGE-B6, MAGE-C1, MAGE-C2, mucin 1 (MUC1), MART-1/Melan-A (MLANA), gp100, gp100/Pme117 (S1LV), tyrosinase (TYR), TRP-1, HAGE, NA-88, NY-ESO-1, NY-ESO-1/LAGE-2, SAGE, Sp17, SSX-1,2,3,4, TRP2-1NT2, carcino-embryonic antigen (CEA), Kallikfein 4, mammaglobm-A, OA1, prostate specific antigen (PSA), prostate specific membrane antigen, TRP-1/gp75, TRP-2, adipophilin, interferon inducible protein absent in nielanorna 2 (AIM-2), BING-4, CPSF, cyclin D1, epithelial cell adhesion molecule (Ep-CAM), EpbA3, fibroblast growth factor-5 (FGF-5), glycoprotein 250 (gp250intestinal carboxyl esterase (iCE), alpha-feto protein (AFP), M-CSF, mdm-2, MUCI, p53 (TP53), PBF, FRAME, PSMA, RAGE-1, RNF43, RU2AS, SOX10, STEAP1, survivin (BIRCS), human telomerase reverse transcriptase (hTERT), telomerase, Wilms' tumor gene (WT1), SYCP1, BRDT, SPANX, XAGE, ADAM2, PAGE-5, LIP1, CTAGE-1, CSAGE, MMA1, CAGE, BORIS, HOM-TES-85, AF15q14, HCA66I, LDHC, MORC, SGY-1, SPO11, TPX1, NY-SAR-35, FTHLI7, NXF2 TDRD1, TEX 15, FATE, TPTE, immunoglobulin idiotypes, Bence-Jones protein, estrogen receptors (ER), androgen receptors (AR), CD40, CD30, CD20, CD19, CD33, CD4, CD25, CD3, cancer antigen 72-4 (CA 72-4), cancer antigen 15-3 (CA 15-3), cancer antigen 27-29 (CA 27-29), cancer antigen 125 (CA 125), cancer antigen 19-9 (CA 19-9), beta-human chorionic gonadotropin, 1-2 microglobulin, squamous cell carcinoma antigen, neuron-specific enoJase, heat shock protein gp96, GM2, sargramostim, CTLA-4, 707 alanine proline (707-AP), adenocarcinoma antigen recognized by T cells 4 (ART-4), carcinoembryogenic antigen peptide-1 (CAP-1), calcium-activated chloride channel-2 (CLCA2), cyclophilin B (Cyp-B), human signet ring tumor-2 (HST-2), Human papilloma virus (HPV) proteins (HPV-E6, HPV-E7, major or minor capsid antigens, others), Epstein-Barr vims (EBV) proteins (EBV latent membrane proteins-LMP1, LMP2; others), Hepatitis B or C virus proteins, and HIV proteins.

### V. ARTICLES OF MANUFACTURE OR KITS

An article of manufacture or a kit is provided comprising at least one immune checkpoint inhibitor (e.g., anti-PD-1 antibody and/or anti-CTlA-4 antibody) and an anti-sMIC antibody or antigen-binding fragment thereof is also provided herein. The article of manufacture or kit can further comprise a package insert comprising instructions for using the at least one checkpoint inhibitor in conjunction with a tumor suppressor gene therapy to treat or delay progression of cancer in an individual or to enhance immune function of an individual having cancer. Any of the immune checkpoint inhibitor and anti-sMIC antibody or antigen-binding fragment thereof, described herein may be included in the article of manufacture or kits. The kit may additionally comprise an extracellular matrix degrading protein or expression construct encoding the extracellular matrix degrading protein.

In some embodiments, the at least one immune checkpoint inhibitor (e.g., anti-PD-1 antibody and/or anti-CTLA-4 antibody) and an anti-sMIC antibody or antigen-binding fragment thereof are in the same container or separate containers. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and antineoplastic agent). Suitable containers for the one or more agent include, for example, bottles, vials, bags and syringes.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Combination of Anti-sMIC Antibody and Anti-CTLA-4

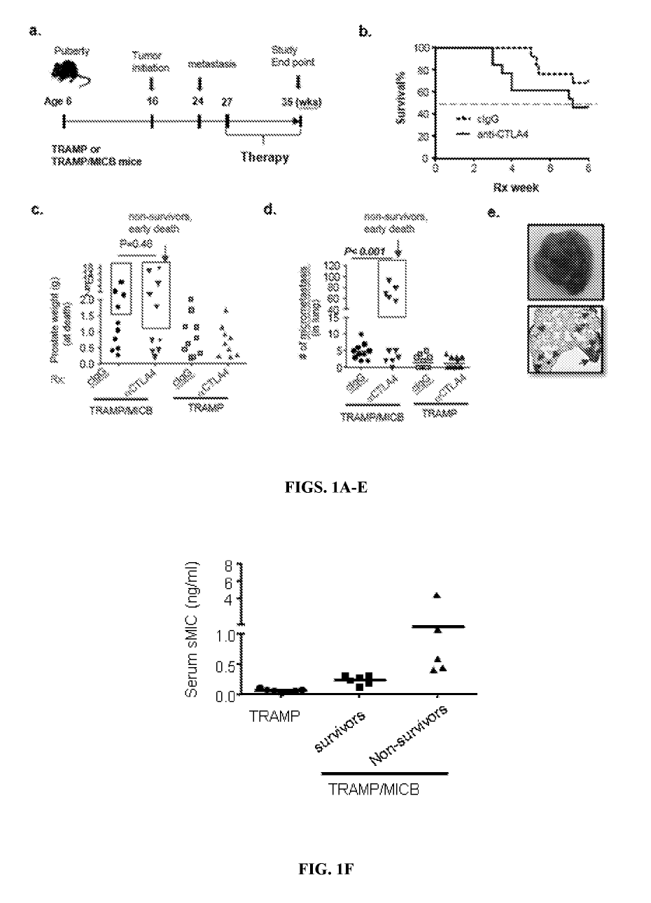

High Serum Levels of sMIC induce adverse response to anti-CTLA4 therapy: Clinical case studies have demonstrated that cancer patients who developed anti-sMIC autoantibody during the course of anti-CTLA4 therapy elicited better clinical response (Jinushi et al., 2006), which suggests that sMIC may negatively affect the response to anti-CTLA4 therapy. However, due to the facts that rodents do not express human NKG2D ligand MIC and shedding of MIC to evade immune surveillance is a clinic manifestation. Thus, the clinically relevant MIC-humanized bi-transgenic TRAMP/MIC prostate tumor mouse model was used for the current investigation (Liu et al., 2013). Akin to human malignancies, in this model, MIC and the oncogene SV4OT were concurrently expressed in male mice upon puberty. Furthermore, tumor shedding sMIC correlates with disease stages. To address the impact of sMIC on the efficacy of anti-CTLA4 therapy, cohorts of age-matched TRAMP and TRAMP/MIC mice were treated with anti-CTLA4 antibody (3.0 mg/kg) or control IgG (FIG. 1A). Consistent with other studies (Hurwitz et al., 2006; Wada et al., 2013), TRAMP mice presented only marginal response to anti-CTLA4 monotherapy (FIGS. 1B-1D). Intriguingly, 40% (4/10) of TRAMP/MIC mice elicited severe adverse response to anti-CTLA4 therapy and succumbed to disease progression with 3-4 weeks of receiving therapy (FIGS. 1B-1D). The adverse response to CTLA4 therapy presented as large primary tumors and massive lung metastasis (FIGS. 1C-1D). The animals that had high levels of sMIC and received anti-CTLA4 therapy also developed colitis such as inflammation in the colon but no obvious diarrhea (FIG. 8). These data indicated that high levels of circulating sMIC adversely impact tumor response to anti-CTLA4 therapy and also contribute, at least in part, to anti-CTLA4-induced autoimmune colitis.

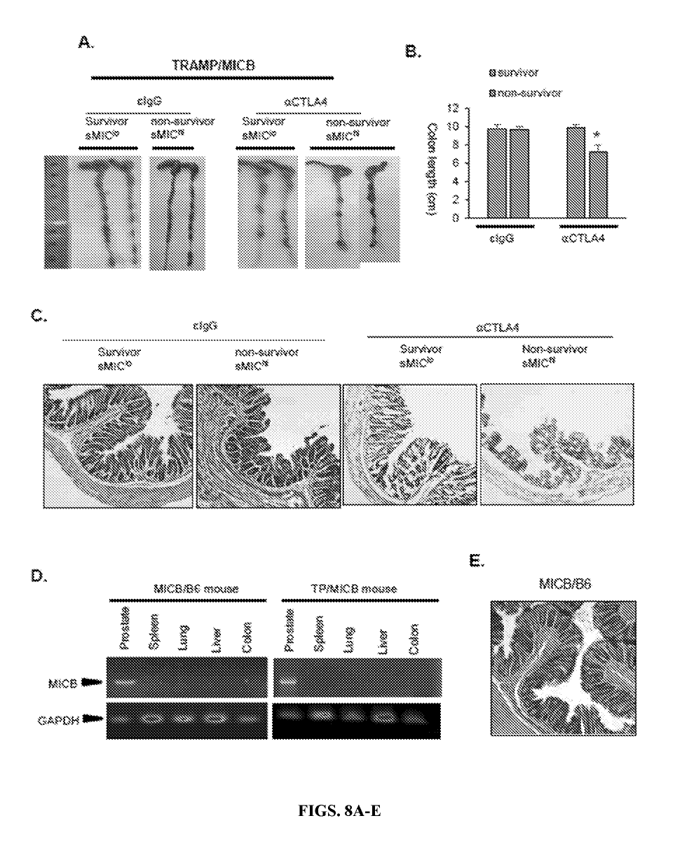

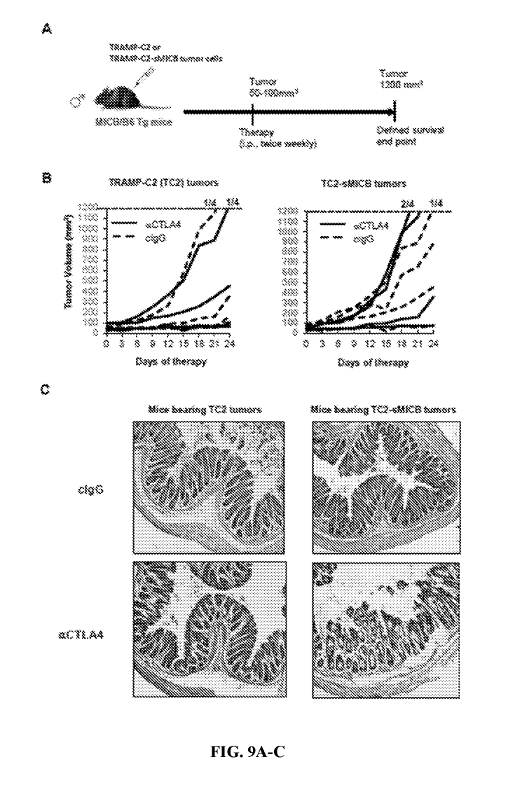

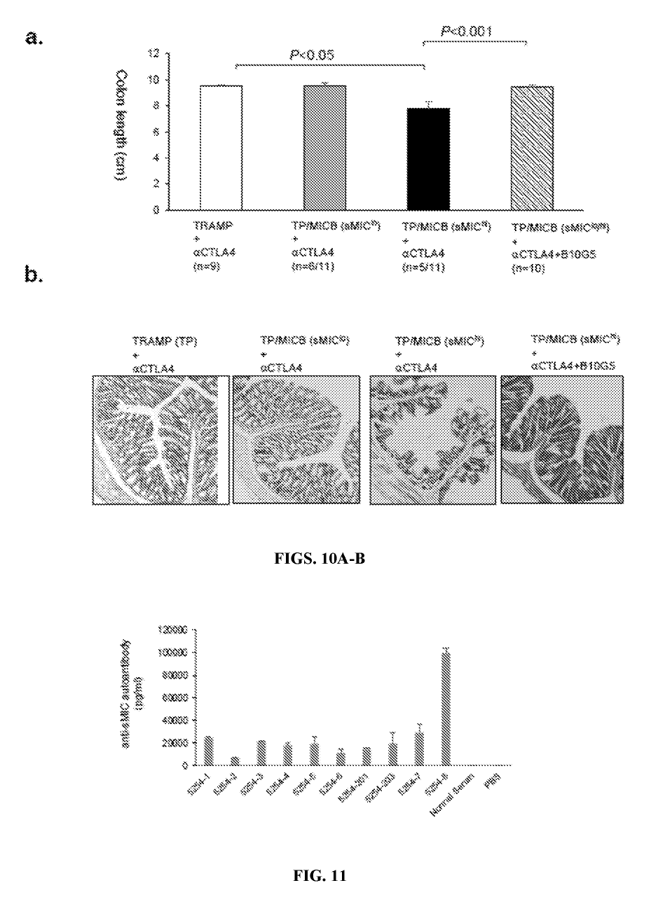

It was then investigated whether expression of MICB in the prostate would provoke worsening diseases in response to anti-CTLA4 therapy in TRAMP/MICB mice. As shown in FIG. 1E, prior to the therapy, all TRAMP/MICB animals that presented negative response to anti-CTLA4 and succumbed to disease (i.e., non-survivors) had significantly higher levels of serum sMIC than those that survived to the study end point (i.e., survivors). The observations were substantiated in syngeneic TRAMP-C2 and sMICB-expressing TRAMP-C2-sMICB transplantable prostate tumor models (FIG. 9), where both tumor cells lines were inoculated subcutaneously into MICB/B6 male transgenic mice. In the absence of sMIC, TRAMP-C2 tumors presented no response to anti-CTLA4 therapy. Consistent with observations in TRAMP/MICB mice, TRAMP-C2-sMICB tumors grew more aggressively in response to anti-CTLA4 therapy (FIG. 9B). In animals that received anti-CTLA4 therapy, TRAMP-C2-sMICB tumor-bearing mice developed more severe colon inflammation than TRAMP-C2 tumor bearing mice (FIG. 9C).

B10G5 Antibody Neutralizing sMIC Generate Cooperative Therapeutic Effect with Anti-CTLA4 Therapy:

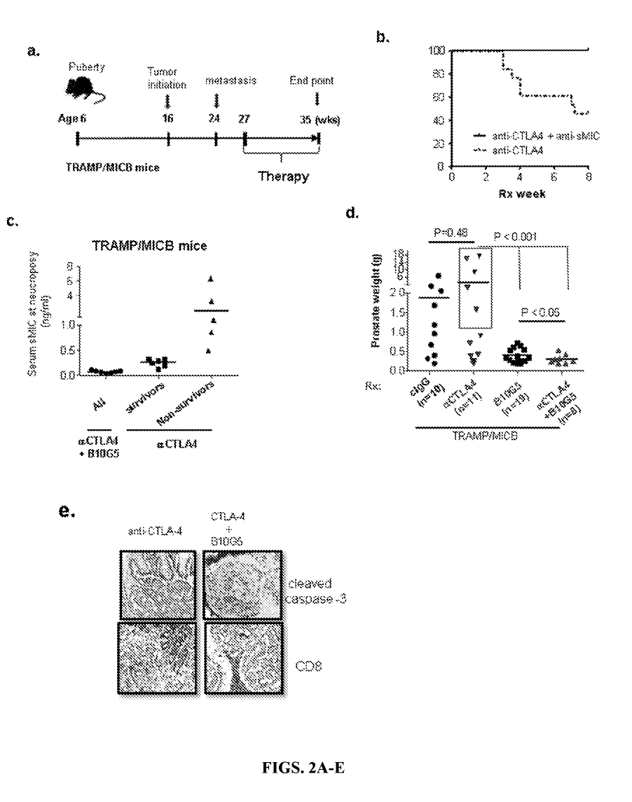

In has been shown that neutralizing sMIC with a non-blocking anti-sMIC antibody B10G5 effectively not only restores NKG2D expression on CD8⁺ T cells but also revamps CD8 T cell function (Lu et al., 2015). To confirm that sMIC attributes to the adverse response to anti-CTL4 therapy and to further investigate whether neutralizing sMIC will enhance the therapeutic efficacy of anti-CTLA4 for MIC-positive tumors, cohorts of TRAMP/MICB mice of 28-week of age when at least 50% of the animals had developed advanced diseases (Liu et al., 2103; Lu et al., 2015), with anti-sMIC mAb B10G5 (3.0 mg/Kg), anti-CTLA4 (3.0 mg/Kg), or a cocktail containing both antibodies (FIG. 2A). Nearly all animals responded to mAb B10G5 anti-sMIC monotherapy. As presented above, animals with high serum sMIC elicited an adverse response to anti-CTLA4 monotherapy. However, regardless of serum levels of sMIC prior to receiving therapy (FIG. 2B), all animals elicited a favorable response to the cocktail therapy of anti-CTLA4 and anti-sMIC with 100% survival to the study endpoint (FIG. 2C). As a result of the cocktail therapy, circulating sMIC was significantly eliminated from all animals (FIG. 2B). Remarkably, cocktail therapy of anti-CTLA4 and B10G5 anti-sMIC generated cooperative therapeutic effect as demonstrated by prostate weight at the study endpoint (FIG. 2D) and by histological characterization (FIG. 2E). No inflammation in the colon or other organs was observed in animal receiving combined therapy of anti-CTLA4 and anti-sMIC (FIG. 8). These data have not only confirmed that high levels of sMIC is a contributing factor to the adverse response during anti-CTLA4 therapy, but also clearly demonstrated the therapeutic benefit of the anti-CTLA4 and anti-sMIC antibody cocktail.

A case was reported that a melanoma patient who developed anti-MIC autoantibody during anti-CTLA4 therapy elicited better response. The presence of anti-sMIC autoantibody during early response to anti-CTLA4 (ipilimumab) Phase I/II clinical trial (NCT01498978) on a small cohort (n=10) of metastatic prostate cancer patients who were also receiving androgen suppression therapy. High titer of anti-MIC autoantibody was detected in one patient after 1 cycle of ipilimumab (FIG. 11). The patient elicited durable response with PSA from 191 mg/ml to 4.6 ng/ml after 8 cycles of ipilimumab. This case study further signifies the utility of sMIC-neutralizing antibody in enhancing the therapy of anti-CTLA4.

Antibody Neutralizing sMIC and Anti-CTLA4 Therapy Cooperatively Heightens CD8 T Cell Anti-Tumor Potential:

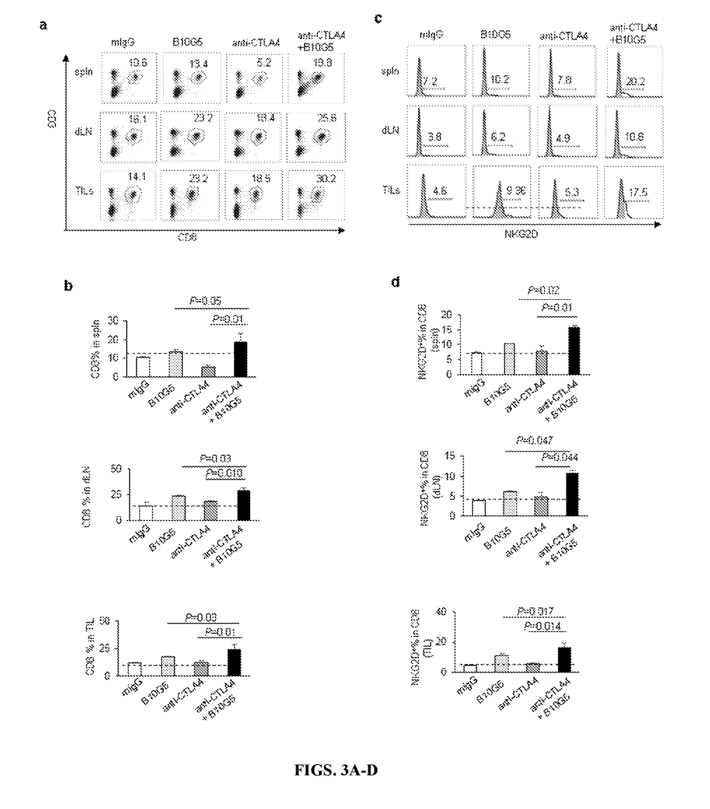

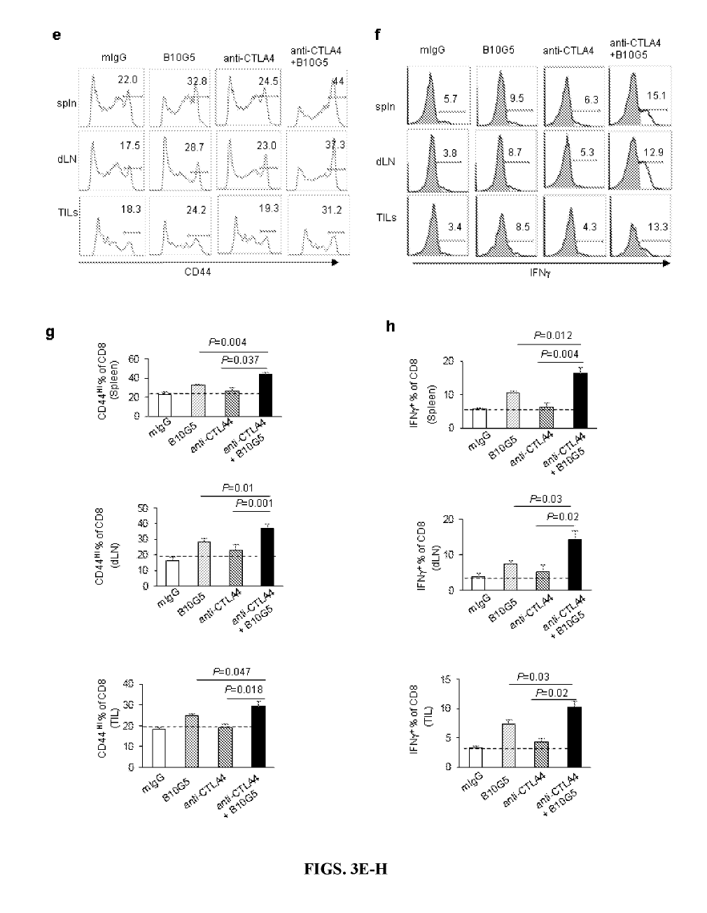

The mechanisms that are associated with the cooperative therapeutic effect of B10G5 anti-sMIC and anti-CTLA4 were investigated next. Data was not collected from animals that succumbed to a large tumor burden and metastasis before the experimental end point. Of all the animals that survived to the experimental end point, therapy with the combined antibody cocktail resulted in a significant increase of CD8⁺ T cells in tumor draining lymph nodes (dLN) and tumor infiltrates in comparison to monotherapy with anti-CTLA4 or B10G5 (FIG. 3A, 3B). As with what has been previously shown, B10G5 therapy alone restores the expression of NKG2D, the co-stimulatory receptor on CD8⁺ T cells (Lu et al., 2015). Antibody cocktail therapy of anti-CTLA4 and B10G5 anti-sMIC further increased the expression of NKG2D on CD8⁺ T cells (FIG. 3C, 3D). This increased expression of NKG2D provides a co-stimulatory signal to augment CD8⁺ T cell response to tumors that in part retained surface MIC expression, in addition to the diminution of CTL4 co-inhibitory signals.

The heightened CD8⁺ T cell anti-tumor potential with B10G5 and anti-CTLA4 cocktail therapy was further demonstrated by the significant increase in effector memory-like CD44^{bi} CD8⁺ T cell compartments in the spleen, dLN, and tumor infiltrates (FIGS. 3E, 3F) and the ability to produce IFNγ in response to PMA and ionomycin stimulation (FIGS. 3G, 3H).

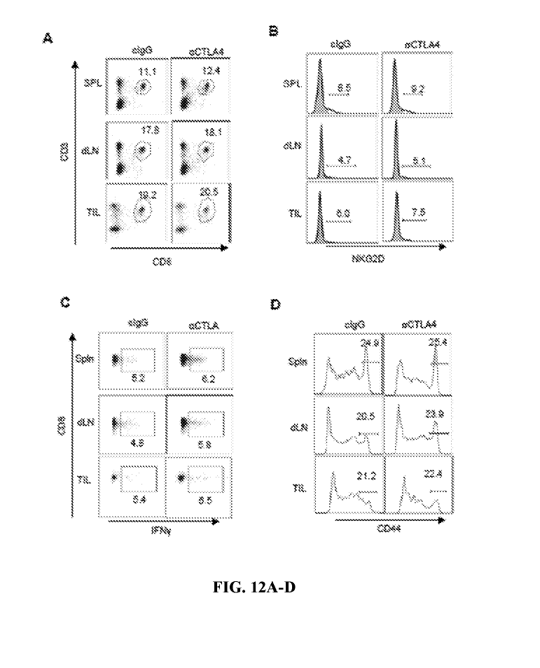

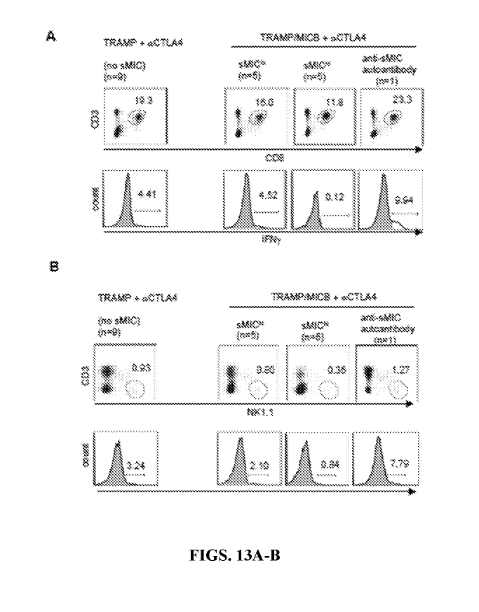

The functional potential of CD8 T cells from anti-CTLA4-treated TRAMP and TRAMP/MICB mice was retrospectively compared. CD8 T cells in all TRAMP mice uniformly exhibited nominal response to anti-CTLA therapy (FIG. 11). Interestingly, in anti-CTLA4-treated TRAMP/MIC mice, CD8 T cell response to ex vivo PMA/I re-stimulation varied in accordance to serum levels of sMIC, although the percentage of CD8 T cells was not profoundly impacted (FIG. 12A). A severely impaired response was seen in mice with high levels of sMIC, whereas the response in TRAMP/MICB mice with low levels of sMIC and in TRAMP mice remain comparable. Intriguingly, one of the 11 TRAMP/MICB mice that developed anti-sMIC autoantibody during anti-CTLA4 monotherapy demonstrated remarkable responsiveness to ex vivo re-stimulation. Notably, the polarization of CD8 T cell functional potential was correlative with the presence and function of NK cells in the tumor-draining lymph nodes. Given that sMIC perturbs NK cell homeostatic maintenance and function and that NK-DC cross-talk are critical in regulating T cell priming, these observations further highlight a potential important role of NK cells in effectuating CTLA4 blockade therapy.

Antibody Neutralizing sMIC and Anti-CTLA4 Therapy Cooperatively Overcomes Antigen-Specific CD8 T Cell Tolerance:

To further address whether B10G5 anti-sMIC and anti-CTLA4 antibody cocktail therapy can enhance CD8⁺ T cell anti-tumor response in an antigen-specific manner, CFSE-labeled CD8⁺ T cells isolated from TCR-I transgenic mice were adoptively transferred into cohorts of mice that were under antibody therapies as depicted in FIG. 2A. These CD8⁺ T cells bear TCR specific for the TRAMP/MICB oncogene SV4OT and can be detected by SV40TAg peptide I-specific D^{b}/I-tetramer. The sustainability of these TCR-1 CD8⁺ T cells was analyzed on day 14 post-adoptive transfer. Generally, adoptively transferred antigen-specific TCR-I CD8⁺ T cells are not able to sustain in TRAMP or TRAMP/MICB mice due to clonal deletion after initial expansion (Lu et al., 2015; Bai et al., 2008).

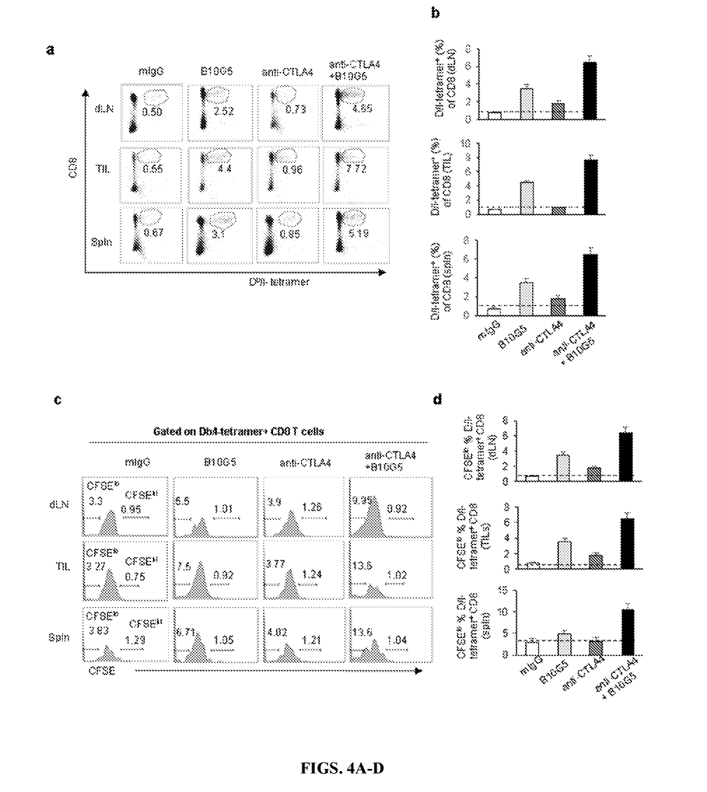

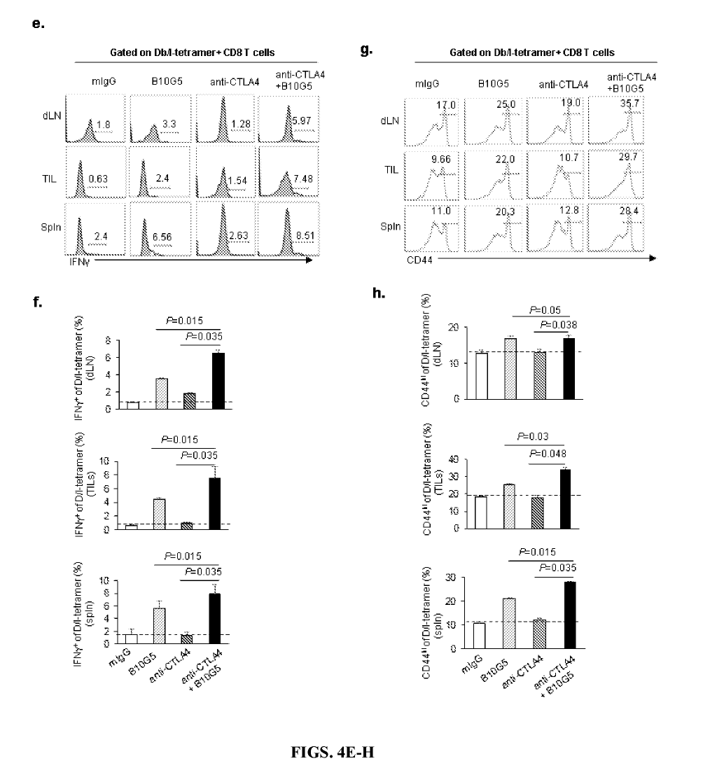

Anti-CTLA4 monotherapy only had a marginal effect in comparison to control IgG therapy on sustaining the D^{b}/I-tetramer⁺ TCR-1 CD8⁺ T cells in dLN, tumor infiltrates or spleen (FIG. 4A, 4B). However, B10G5 anti-sMIC therapy markedly enhanced the sustainability of adoptively transferred TCR-1 CD8⁺ T cells with a great percentage in the tumor infiltrate. Remarkably, antibody cocktail therapy with B10G5 anti-sMIC and anti-CTLA4 further significantly enhanced SV40TAg-specific TCR-I CD8⁺ T cells in tumor infiltrates, and spleen in comparison to B10G5 monotherapy (FIG. 4A, 4B).

CFSE dilution assay confirmed that adoptively transferred naïve D^{b}/I-tetramer⁺ TCR-1 CD8⁺ T cells underwent initial expansion (shown as dilution of CFSE^{hi} D^{b}/I-tetramer⁺ CD8 T cells in FIG. 4C, 4D) upon encounter antigen in all cohorts of animals. However, only therapy with mAb B10G5 or mAb cocktail of B10G5 and anti-CTLA4 provoked continuous expansion of SV40TAg-specific TCR-1 CD8⁺ T cells, under which provision mAb cocktail therapy remarkably enhanced the expansion in comparison to B10G5 therapy alone (nearly doubled, as shown by % of CFSE^{lo} D^{b}/I-tetramer⁺ CD8⁺ T cells in FIGS. 4C, 4D).

Continuous expansion of antigen-specific CD8⁺ T cells in the tumor microenvironment and tumor-draining lymph nodes is considered a positive outcome and an indication of the activation of antigen-specific CD8⁺ T cells. The cocktail therapy of mAb B10G5 and anti-CTLA4 resulted in remarkable a cooperative increase in IFNγ production by D^{b}/I-tetramer⁺ CD8 T cells in response to PMA/I stimulation (FIGS. 4E, 4F) and the number of CD44^{hi}D^{b}/I-tetramer⁺ CD8⁺ T cells in tumor infiltrates (FIGS. 4G, 4H). Together, this data have demonstrated that therapy with antibody cocktail of B10G5 anti-sMIC and anti-CTLA4 cooperatively and synergistically heightened antigen-specific CD8⁺ T cell anti-tumor responses, beyond an additive effect.

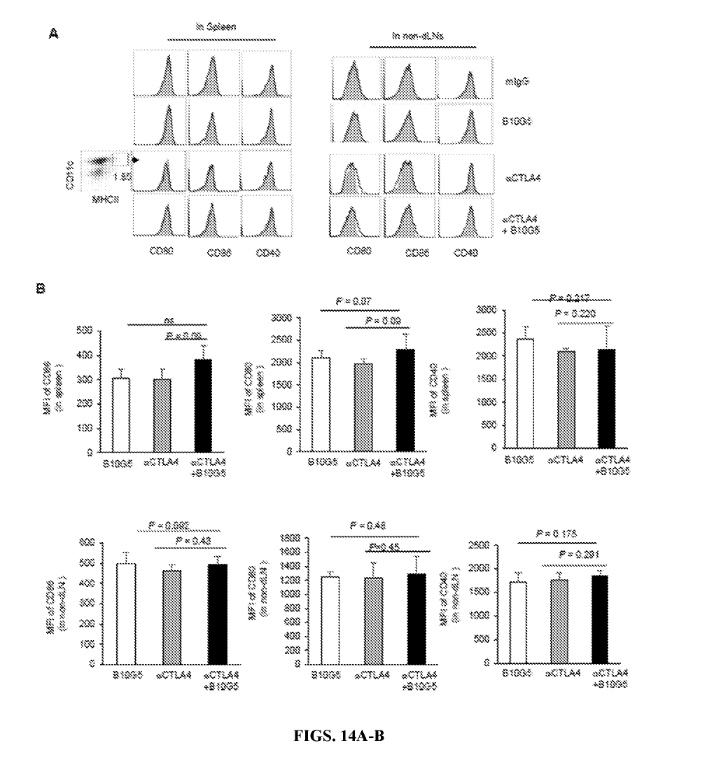

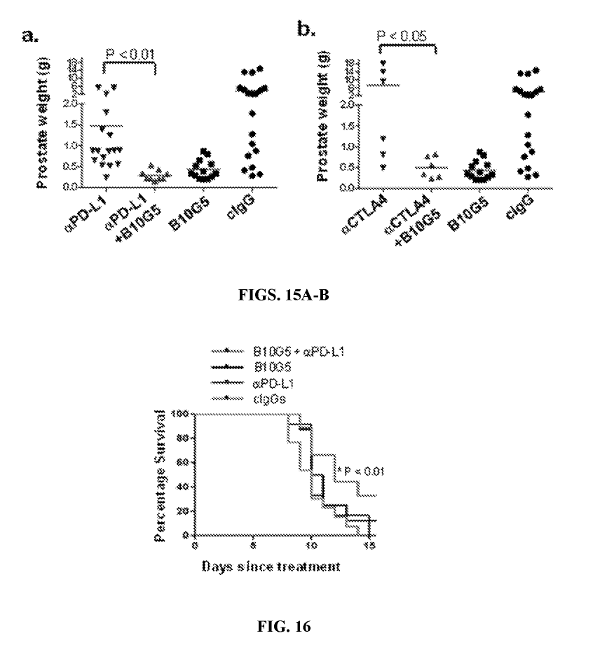

B10G5 neutralizing sMIC and anti-CTLA4 cooperatively heightens the co-stimulatory potential of dendritic cells: The mechanisms whereby the cocktail therapy of B10G5 anti-sMIC and anti-CTLA4 would synergistically bolster antigen-specific CD8⁺ T cell responses was investigated. It has been previously shown that B10G5 therapy alone can enhance DC activation in tumor draining lymph nodes and increase the expression of DC co-stimulatory molecules CD80 and CD86 (Lu et al., 2015). With the cocktail antibody therapy, a further significant increase in DC surface CD80 and CD86 was achieved in tumor draining lymph nodes and tumor infiltrates (FIGS. 5A, 5B). Such an effect was not observed in non-tumor draining lymph nodes (FIG. 14). Given that CD80 and CD86 can engage both the co-stimulatory molecule CD28 and the co-inhibitory molecule CTLA4, the data provided a reasonable explanation that blocking the engagement of CTLA4 would allow a more robust CD28-mediated co-stimulatory signal delivered to antigen-specific CD8⁺ T cells which may in part contribute to the synergistic effect.

Anti-sMIC Therapy Increases T Cell TCR Clonality and Repertoire Complexity:

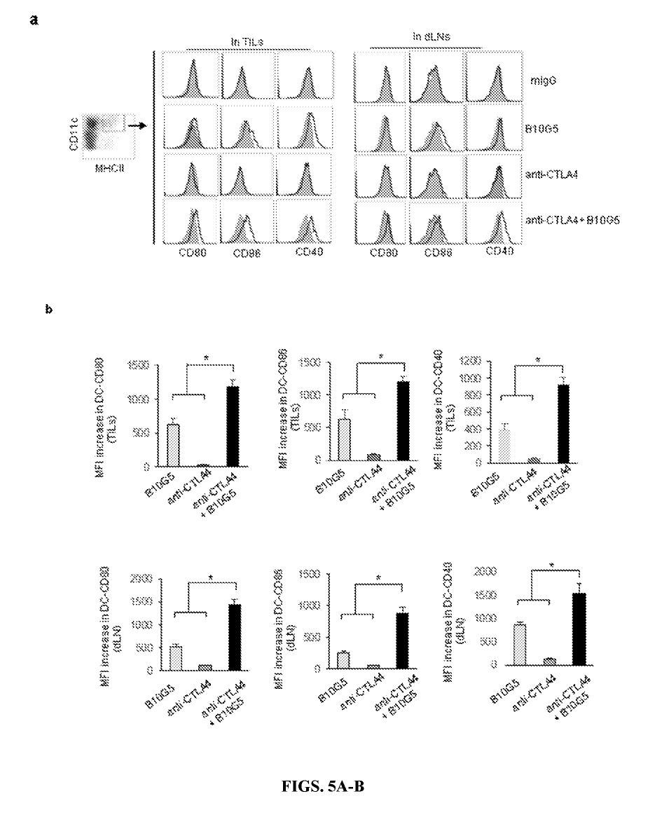

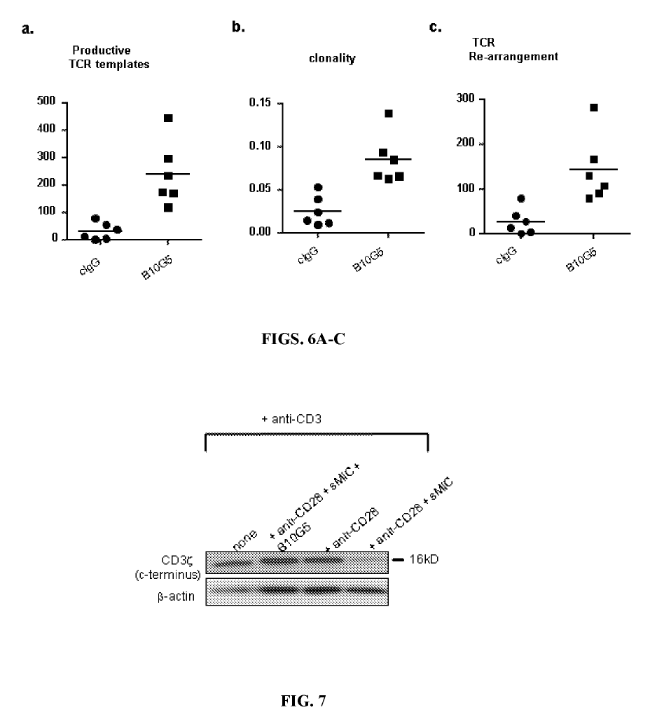

It has been reported that the clinical response to anti-CTLA4 therapy correlates to TCR clonal stability and repertoire complexity (Cha et al., 2014; Robert et al., 2014). In general, better clinical outcome in response to immunotherapy is found to be associated with higher clonal expansion of CD8⁺ T cells and complexity of TCR repertoire in patients (Cha et al., 2014; Robert et al., 2014; Sheikh et al., 2016; Tumeh et al., 2014). Thus, the impact of sMIC on TCR repertoire was addressed in tumor infiltrates. It has been shown previously that anti-sMIC therapy markedly increases the number and activation status of CD8⁺ T cells in tumors. Surveying TCR sequences within the prostate tumor tissues using in situ ImmunSEQ technology revealed that B10G5 neutralizing sMIC remarkable increased TCR clonality, presumably due to clonal expansion (Lu et al., 2015), and TCR repertoire diversity in tumor infiltrates (FIGS. 6A, 6B). The broadening of the TCR repertoire within the prostate tissue indicates that B10G5 therapy facilitates the recruitment of T cells into the tumors.

B10G5 Therapy Stabilizing CD3; in CD8 T Cells:

The mechanisms whereby anti-sMIC therapy increases TCR clonality and thereby enhances the efficacy to anti-CTLA4 therapy was investigated. It has been shown in vitro that sMIC not only down-modulates NKG2D expression but also impairs TCR/CD3 complex signaling of CD8⁺ T cells by de-stabilizing its key downstream signaling molecule CD3ζ (Hanaoka et al., 2010). Thus, intracellular levels of CD3ζ were analyzed in splenic CD8⁺ T cells from the cohorts of experimental animals by flow cytometry. As demonstrated in FIG. 7A, splenic CD8⁺ T cells from TRAMP/MIC mice that had high levels of circulating sMIC had diminished intracellular CD3ζ with anti-CTLA4 therapy. All TRAMP/MICB mice that received B10G5 and anti-CTLA4 therapy had increase in intracellular CD3ζ in CD8 T cells. A more pronounced increase in levels of CD3ζ was shown in prostate tissues with B10G5 anti-sMIC therapy (FIG. 7B). These data indicate that stabilizing CD3ζ to sustain TCR activation by B10G5 therapy may in part account for the clonal expansion of antigen-specific T cells and increased TCR clonality in tumor infiltrates.

Therapy with B10G5 Enhances Responses to Immune Checkpoint Blockade:

With the TRAMP/MIC spontaneous tumor model, it was further demonstrated that B10G5 therapy enhanced the therapeutic efficacy of immune checkpoint blockade of anti-PD-L1 antibody and anti-CTLA4 therapy in late stage of disease. Late stage TRAMP/MIC mice that have developed advanced carcinoma showed no or nominal responses to anti-PD-L1 or anti-CTLA4 therapy (FIG. 11). Remarkably, combined therapy with B10G5 evoked strong responsiveness of TRAMP/MIC mice to anti-PD-L1 or anti-CTLA4 therapy (FIG. 11). These findings were further confirmed with the transplantable syngeneic B16-sMICB model (FIG. 12). Mechanistically, B10G5 therapy induced active immune responses which enabled the effect of unleashing checkpoint blockade.

In a clinical trial with castration-resistant prostate cancer patients, there were ipilimumab toxicity related deaths, although some subgroups of patients have benefited from ipilimumab. Of all the treatment toxicity-related clinical manifestations, IREAS of colitis is most frequent and severe, even death-related. Currently, there is no understanding or relevant biomarker to predict the patient population who will elicit an adverse reaction to ipilimumab therapy. The unexpected findings of the above studies in TRAMP/MIC mice suggest that high levels of tumor-derived sMIC may be one of the co-founders and also indicators for anti-CTLA4 therapy-induced autoimmune colitis in MIC⁺ cancer patients. It is notewothy that animals with high levels of circulating sMIC developed severe colon inflammation or colitis in response to anti-CTLA4 therapy and that antibody neutralizing sMIC alleviated this adverse autoimmune effect.

Thus, the clinically relevant TRAMP/MICB double transgenic mouse model demonstrated that the antibody cocktail co-targeting soluble NKG2D ligand sMIC not only remarkable augmented therapeutic efficacy of immune checkpoint blockade anti-CTLA4 or PD-1 but also generated cooperative therapeutic effect with immune checkpoint inhibitor therapy. Cp-targeting sMIC and CTLA4 cooperatively primed DC activation and upregulated expression of CD80/CD86 co-stimulatory molecule on DCs, overcame CD8 T cell tolerance, enhanced TCR/CD3 signaling capacity in CD8 T cells, and increased T cell clonality or repertoire complexity in tumor infiltrates. Thus, the antibody co-targeting soluble NKG2D sMIC and immune checkpoint inhibitor therapy can generate cooperative therapeutic effect.

### Example 2—Materials and Methods

Antibodies and Flow Cytometry:

Single cell suspension from spleens, tumor draining lymph nodes (dLN), non-dLN, or tumor tissues were prepared as previously described (Lu et al., 2015). Combinations of the following fluorochrome-conjugated antibody were used for cell surface or intracellular staining to define populations of NK, CD8, and subsets of CD4 T cells: CD3e (clone 145-2c11), CD8a (clone 53-6.7), CD4 (clone GK1.5), NK1.1 (clone PK136), NKG2D (clone CX5), CD44 (clone eBio4B10), CD11c (clone N418), MHCII (clone M5/114.15.2), CD80 (clone 16-10A1), CD86 (clone P03) and CD40 (clone 1C10). For ex vivo re-stimulation, freshly isolated single cell suspension was cultured in complete RPMI 1640 medium containing 50 ng/mL PMA and 500 ng/mL Ionomycin for 6 h before being analyzed for IFNγ production by intracellular staining with an antibody specific to IFNγ (XMG1.2). Multicolored flow cytometry analyses were performed on an LSR II (BD). Data were analyzed with FlowJo software (Tree Star).

Animals and Antibody Therapy:

All animal experimental procedures were approved by the Institutional Animal Care and Use Committee (IACUC) at MUSC. Male TRAMP mice at the age of 27-28 weeks old were randomized into two treatment cohorts: 1) control IgG (3 mg/KG BW); or 2) anti-CTLA4 (3 mg/KG BW, clone 9H10, in vivo Mab grade, BioXCell). In parallel, age-matched TRAMP/MICB double transgenic mice were randomized into four treatment cohorts: 1) anti-sMIC mAb B10G5 (3 mg/KG) (Liu et al., 2013); 2) anti-CTLA4 (3 mg/KG; 9H10); 3) cocktail of B10G5 and anti-CTLA4; 4) cocktail of control IgG. All antibodies were given by i.p. injection twice a week. All animals received the treatment for an eight-week duration before being euthanized at the study endpoint, unless succumbed to diseases or adverse effect at an earlier time point.

Antigen-Specific T Cell Tolerance Experiment:

The experimental procedure was described previously (Lu et al., 2015). Briefly, CD8⁺ T cells from TCR-I transgenic mice were labeled with CFSE and injected i.v. into animals (2×10⁶cells/mouse) in the respective experimental groups. Animals were sacrificed at indicated time points to assess TCR-I T cell in vivo frequency with TCR-I-specific H-2D^{b}/TAg epitope I-tetramer (D^{b}/I-tetramer) (Staveley-O'Carroll et al., 2003). To assay antigen-specific CD8⁺ T cell response, bulked single cell suspension prepared from the spleen, LN, and tumor digests was stimulated overnight with 0.5 μM TAg epitope I peptide (SAINNYAQKL; SEQ ID NO:7) and assayed for intracellular IFNγ expression of CD8⁺ or D^{b}/I-tetramer⁺ T cells by flow cytometry.

Tissue Collection:

Blood was collected via tail bleeding during therapy and via cardiac puncture after euthanization. Splenocytes, draining lymph nodes (dLN), non-draining lymph nodes were collected for immunological analyses. Prostate, lung, liver, kidney, pancreas, and colons were collected and fixed in 10% neutral fixation buffer followed by paraffin embedment or directly embedded in OCT, for pathological and histological analyses. In some experiments, portion of prostate tumors were used for parathion of single cell suspension.

Histology and Immunohistochemistry Staining (IHC):

ALL collected tissues were sectioned and stained with H&E for histological evaluation. For imunohistochemistry staining to detect specific antigens, the following antibodies were used: anti-Ki67 (Neo markers), anti-cleaved Caspase-3 (cell signaling, clone 5A1E), anti-CD8 (BD Biosciences), anti-NK1.1 (eBiosciences, PK136), anti-SV40Tag (Santa Cruz), anti-Arginase I Santa Cruz) and anti-p63 (Neo markers). The IHC staining protocol has been previously described (Liu et al., 2013; Lu et al., 2015). All sections were counter stained with hemotoxyline for visualization of nucleus.

Serum sMIC and Cytokine Detection:

Serum levels of sMICB were assessed using respective Sandwich ELISA kit (R&D systems). Serum levels of cytokines were assayed by Eve Technologies Corporation using the Luminex technology (Alberta, Canada).

High Throughput TCR Sequencing in Tumor Tissues:

High-throughput TCR sequence survey from paraffin-embedded tissue was performed according to the protocol described elsewhere (Sheikh et al., 2016). Briefly, sections from paraffin blocks of the prostate were collected for DNA extraction using the QIAmp DNA FFPE Tissue Kit (Qiagen Inc.), per the manufacturer's instructions. The TCRβ CD3 (CDR3β) region was amplified, sequenced, and quantified from a standardized 1,200 ng of DNA using the ImmunoSEQ assay as described (Adaptive Biotechnologies) (Sheikh et al., 2016; Robins et al., 2009). TCR sequence diversity and clonality were determined by the Shannon diversity index using ImmunoSEQ Analyzer software (Adaptive Biotechnologies) (Sheikh et al., 2016).

Statistical Analysis:

All results are expressed as the mean±SEM, unless specified. Mouse and sample group sizes were n>5, unless otherwise indicated. Data were analyzed using ANOVA unpaired t-test. Differences between means were considered significant at P<0.05. Kaplan-Meier survival curves were generated and analyzed using GraphPad Prism software.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Alexander et al., 1988.
Austin-Ward and Villaseca, 1998.
Bai et al., Rapid tolerization of virus-activated tumor-specific CD8⁺ T cells in prostate tumors of TRAMP mice. Proc Natl Acad Sci USA 105, 13003-13008, 2008.
Bajorin et al., 1988.
Bauer et al., Activation of NK cells and T cells by NKG2D, a receptor for stress-inducible MICA. Science 285, 727-729, 1999.
Bird et al., Science 242, 423-426, 1988.
Bukowski et al., 1998.
Camacho et al., J Clin Oncology 22(145): Abstract No. 2505 (antibody CP-675206), 2004.
Cha et al., Improved survival with T cell clonotype stability after anti-CTLA-4 treatment in cancer patients. Sci Transl Med 6, 238ra270, 2014.
Chen and Okayama, 1987.
Christodoulides et al., 1998.
Churchill Livingstone, Inc; Baltzer L, Berkery R (eds): Oncology Pocket Guide to Chemotherapy, 2nd ed. St. Louis, Mosby-Year Book, 1995.
Davidson et al., 1998.
Dillman, 1999.
Ercolani et al., 1988.
Fechheimer et al., 1987.
Fischer et al., (eds): The Cancer Chemotherapy Handbook, 4th ed. St. Louis, Mosby-Year Book, 1993.
Fraley et al., 1979.
Gopal, 1985.
Graham and Van Der Eb, 1973.
Groh et al., Costimulation of CD8alphabeta T cells by NKG2D via engagement by MIC induced on virus-infected cells. Nat Immunol 2, 255-260, 2001.
Groh et al., Tumour-derived soluble MIC ligands impair expression of NKG2D and T-cell activation. Nature 419, 734-738, 2002.
Hanaoka et al., NKG2D initiates caspase-mediated CD3zeta degradation and lymphocyte receptor impairments associated with human cancer and autoimmune disease. J Immunol 185, 5732-5742, 2010.
Hanibuchi et al., 1998.
Harland and Weintraub, 1985.
Hellstrand et al., 1998.
Hodi et al., Improved survival with ipilimumab in patients with metastatic melanoma. N Engl J Med 363, 711-723, 2010.
Holdenrieder et al., Soluble MICA in malignant diseases. Int J Cancer 118, 684-687, 2006a.
Holdenrieder, et al., Soluble MICB in malignant diseases: analysis of diagnostic significance and correlation with soluble MICA. Cancer Immunol Immunother 55, 1584-1589, 2006b.
Holliger et al., Proc. Natl. Acad. Sci. USA 90:64446448, 1993.
Hui and Hashimoto, 1998.
Hurwitz et al., Proc Natl Acad Sci USA 95(17): 10067-10071, 1998.
Hurwitz et al., Combination immunotherapy of primary prostate cancer in a transgenic mouse model using CTLA-4 blockade. Cancer Res 60, 2444-2448, 2000.
Huston et al., Proc. Natl. Acad. Sci. USA., 85, 5879-5883, 1988.
Irie & Morton, 1986.
Irie et al., 1989.
Jinushi et al., Therapy-induced antibodies to MHC class I chain-related protein A antagonize immune suppression and stimulate antitumor cytotoxicity. Proc Natl Acad Sci USA 103, 9190-9195, 2006.
Jones et al., J Exp Med., 205(12):2763-79, 2008.
Kabat, et al., Sequences of Proteins of Immunological Interest, Fifth Edition, U.S. Department of Health and Human Services, NIH Publication No. 91-3242, 1991.
Kaeppler et al., 1990.
Kaneda et al., 1989.
Karin et al., 1989.
Kato et al., 1991.
Kipriyanov et al., Mol. Immunol. 31: 10471058, 1994.
Kipriyanov et al., Human Antibodies and Hybridomas 6:93-101, 1995.
Lanzavecchia et al., Eur. J Immunol. 17, 105, 1987.
Larkin et al., Combined Nivolumab and Ipilimumab or Monotherapy in Untreated Melanoma. N Engl J Med 373, 23-34, 2015.
Liu et al., Perturbation of NK cell peripheral homeostasis accelerates prostate carcinoma metastasis. The Journal of clinical investigation 123, 4410-4422, 2013.
Lu et al., Non-blocking monoclonal antibody targeting soluble MIC revamps endogenous innate and adaptive anti-tumor responses and eliminates primary and metastatic tumors. Clin Cancer Res, 2015.
Lui et al., Gene Therapy, 10:292-303, 2003.
Markiewicz et al., Costimulation through NKG2D enhances murine CD8+ CTL function:
similarities and differences between NKG2D and CD28 costimulation. J Immunol 175, 2825-2833, 2005.
Marten et al., Soluble MIC is elevated in the serum of patients with pancreatic carcinoma diminishing gammadelta T cell cytotoxicity. Int J Cancer 119, 2359-2365, 2006.
Mellman et al., Nature 480:480-489, 2011.
Mitchell et al., 1990.
Mitchell et al., 1993.
Mokyr et al., Cancer Res 58:5301-5304, 1998.
Morton & Ravindranath, 1996.
Morton et al., 1992.
Nabel et al, 1989.
Ng, 1989.
Nicolau and Sene, 1982.
Nicolau et al., 1987.
Okazaki T et al., Intern. Immun. 2007 19(7):813, 2007.
Pardoll, Nat Rev Cancer, 12(4): 252-64, 2012.
Perry et al., Chemotherapy, Ch. 17 in Abeloff, Clinical Oncology 2^{nd} Edition, 2000.
Pietras et al., 1998.
Poljak et al., Structure 2: 1 121-1 123, 1994.
Potrykus et al., 1985.
Potter et al., 1984.
Qin et al., 1998.
Queen et al., Proc Natl Acad Sci USA 86: 10029-10033, 1989,
Quitsche et al., 1989.
Ravindranath & Morton, 1991.
Richards et al., 1984.
Rippe et al., 1990.
Robert et al., Distinct immunological mechanisms of CTLA-4 and PD-1 blockade revealed by analyzing TCR usage in blood lymphocytes. Oncoimmunology 3, e29244, 2014.
Roberts et al., NKG2D receptors induced by IL-15 costimulate CD28-negative effector CTL in the tissue microenvironment. J Immunol 167, 5527-5530, 2001.
Robins et al., Comprehensive assessment of T-cell receptor beta-chain diversity in alphabeta T cells. Blood 114, 4099-4107, 2009.
Rosenberg et al., 1988; 1989.
Salih et al., Functional expression and release of ligands for the activating immunoreceptor NKG2D in leukemia. Blood 102, 1389-1396, 2003.
Salih et al., Soluble NKG2D ligands: prevalence, release, and functional impact. Front Biosci 13, 3448-3456, 2008.
Serrano-Pertierra et al., NKG2D- and CD28-mediated costimulation regulate CD8+ T cell chemotaxis through different mechanisms: the role of Cdc42/N-WASp. J Leukoc Biol 95, 487-495, 2014.
Sheikh et al., Clonotypic Diversification of Intratumoral T Cells Following Sipuleucel-T Treatment in Prostate Cancer Subjects. Cancer Res 76, 3711-3718, 2016.
Slapak and Kufe, Principles of Cancer Therapy, Chapter 86 in Harrison's Principles of Internal Medicine, 14th edition.
Staveley-O'Carroll et al., In vivo ligation of CD40 enhances priming against the endogenous tumor antigen and promotes CD8+ T cell effector function in SV40 T antigen transgenic mice. J Immunol 171, 697-707, 2003.
Tchekmedyian et al., 2015.
Tsai and Daud, Nivolumab plus ipilimumab in the treatment of advanced melanoma. J Hematol Oncol 8, 123, 2015.
Tumeh et al., PD-1 blockade induces responses by inhibiting adaptive immune resistance. Nature 515, 568-571, 2014.
Tur-Kaspa et al., 1986.
Wada et al., Sequencing CTLA-4 blockade with cell-based immunotherapy for prostate cancer. J Transl Med 11, 89, 2013.
Wang et al., Tumor-derived soluble MICs impair CD3(+)CD56(+) NKT-like cell cytotoxicity in cancer patients. Immunol Lett 120, 65-71, 2008.
Ward et al., (1989) Nature 341:544-546.
Wei et al., E. J. Prospects for the use of ipilimumab in treating advanced prostate cancer. Expert Opin Biol Ther 16, 421-432, 2016.
Wilson et al., 1989.
Wolchok et al., Development of ipilimumab: a novel immunotherapeutic approach for the treatment of advanced melanoma. Ann N Y Acad Sci 1291, 1-13, 2013a.
Wolchok et al., Nivolumab plus ipilimumab in advanced melanoma. N Engl J Med 369, 122-133, 2013b.
Wong et al., 1980.
Wu and Wu, 1987.
Wu and Wu, 1988.
Wu et al., Prevalent expression of the immunostimulatory MHC class I chain-related molecule is counteracted by shedding in prostate cancer. The Journal of clinical investigation 114, 560-568, 2004.
Xiao et al., Soluble NKG2D ligand promotes MDSC expansion and skews macrophage to the alternatively activated phenotype. J Hematol Oncol 8, 13, 2015.
WO 1990/07861
WO 1992/22653
WO 1994/09699
WO 1995/001994
WO 1995/06128
WO 1998/042752
WO 2000/037504
WO 2001/014424
WO 2003/063792
WO 2005/003168
WO 2005/009465
WO 2006/003179
WO 2006/072625
WO 2006/072626
WO 2006/121168
WO 2007/042573
WO 2008/132601
WO 2008/084106
WO 2009/0044273
WO 2009/1 01 611
WO 2009/114335
WO 2010/027827
WO 2010/065939
WO 2011/014438
WO 2011/066342
WO 2012/071411
WO 2012/160448
WO 2013/006490 A2
WO 2013/025779
WO 2013/067492
WO 2015/003114
WO 2015/016718
EP 2320940
U.S. Pat. No. 5,225,539
U.S. Pat. No. 5,302,523
U.S. Pat. No. 5,322,783
U.S. Pat. No. 5,384,253
U.S. Pat. No. 5,464,765
U.S. Pat. No. 5,530,101
U.S. Pat. No. 5,538,877
U.S. Pat. No. 5,538,880
U.S. Pat. No. 5,550,318
U.S. Pat. No. 5,563,055
U.S. Pat. No. 5,563,055
U.S. Pat. No. 5,580,859
U.S. Pat. No. 5,585,089
U.S. Pat. No. 5,589,466
U.S. Pat. No. 5,591,616
U.S. Pat. No. 5,610,042
U.S. Pat. No. 5,656,610
U.S. Pat. No. 5,693,761
U.S. Pat. No. 5,693,762
U.S. Pat. No. 5,702,932
U.S. Pat. No. 5,736,167
U.S. Pat. No. 5,736,524
U.S. Pat. No. 5,739,169
U.S. Pat. No. 5,780,448
U.S. Pat. No. 5,789,215
U.S. Pat. No. 5,801,005
U.S. Pat. No. 5,821,337
U.S. Pat. No. 5,824,311
U.S. Pat. No. 5,830,880
U.S. Pat. No. 5,844,905
U.S. Pat. No. 5,846,945
U.S. Pat. No. 5,885,796
U.S. Pat. No. 5,945,100
U.S. Pat. No. 5,981,274
U.S. Pat. No. 5,994,624
U.S. Pat. No. 6,207,156
U.S. Pat. No. 7,060,808
U.S. Pat. No. 7,223,593
U.S. Pat. No. 7,537,924
U.S. Pat. No. 8,008,449
U.S. Pat. No. 8,017,114
U.S. Pat. No. 8,119,129
U.S. Pat. No. 8,329,660
U.S. Pat. No. 8,329,867
U.S. Pat. No. 8,354,509
U.S. Pat. No. 8,735,553
U.S. Pub. No. 2011/0008369
U.S. Pub. No. 2012/0177645
U.S. Pub. No. 2012/0294796
U.S. Pub. No. 2014/022021
U.S. Pub. No. 2014/0294898

What is claimed is:

1. A method of treating cancer in a subject comprising administering an effective amount of: (a) an anti-soluble MIC (sMIC) antibody or antigen-binding fragment thereof and (b) at least one immune checkpoint inhibitor to the subject.

2. The method of claim 1, wherein the anti-sMIC antibody or antigen-binding fragment thereof is a monoclonal antibody, a chimeric antibody, a CDR-grafted antibody, a humanized antibody, a Fab, a Fab′, a F(ab′)2, a Fv, or a scFv.

3. The method of claim 1, wherein the anti-sMIC antibody is a monoclonal antibody.

4. The method of claim 3, wherein the monoclonal antibody is B10G5 monoclonal antibody.

5. The method of any one of claims 1-4, wherein the at least one checkpoint inhibitor is selected from an inhibitor of CTLA-4, PD-1, PD-L1, PD-L2, LAG-3, BTLA, B7H3, B7H4, TIM3, KIR, or A2aR.

6. The method of any one of claims 1-4, wherein the at least one immune checkpoint inhibitor is a human programmed cell death 1 (PD-1) axis binding antagonist.

7. The method of claim 6, wherein the PD-1 axis binding antagonist is selected from the group consisting of a PD-1 binding antagonist, a PDL1 binding antagonist and a PDL2 binding antagonist.

8. The method of claim 6, wherein the PD-1 axis binding antagonist is a PD-1 binding antagonist.

9. The method of claim 7, wherein the PD-1 binding antagonist inhibits the binding of PD-1 to PDL1 and/or PDL2.

10. The method of claim 7, wherein the PD-1 binding antagonist is a monoclonal antibody or antigen binding fragment thereof.

11. The method of claim 7, wherein the PD-1 binding antagonist is nivolumab, pembrolizumab, pidillizumab, AMP-514, REGN2810, CT-011, BMS 936559, MPDL328OA or AMP-224.

12. The method of any one of claims 1-4, wherein the at least one immune checkpoint inhibitor is an anti-CTLA-4 antibody.

13. The method of claim 12, wherein the anti-CTLA-4 antibody is tremelimumab or ipilimumab.

14. The method of any one of claims 1-4, wherein the at least one immune checkpoint inhibitor is an anti-killer-cell immunoglobulin-like receptor (KIR) antibody.

15. The method of claim 14, wherein the anti-MR antibody is lirilumab.

16. The method of claim 1, wherein the immune checkpoint inhibitor is a recombinant oncolytic virus.

17. The method of claim 16, wherein the recombinant oncolytic virus comprises an expression cassette encoding a soluble form of programmed cell death protein 1 (PD1).

18. The method of claim 16, wherein the virus is replication competent.

19. The method of claim 17, wherein the expression cassette is under the control of a viral promoter.

20. The method of claim 19, wherein the viral promoter is synthetic early/late poxvirus promoter.

21. The method of claim 16, wherein the virus is selected from the group consisting of myxoma virus, reovirus, herpes simplex virus, Newcastle Disease virus, measles virus, retrovirus, poxvirus, rhabdovirus, picornavirus, coxsackievirus and parvovirus.

22. The method of claim 16, wherein the oncolytic virus is myxoma virus.

23. The method of any one of claims 1-22, wherein the cancer is a MIC-positive cancer.

24. The method of any one of claims 1-23, wherein the cancer is melanoma, non-small cell lung, small-cell lung, lung, hepatocarcinoma, retinoblastoma, astrocytoma, glioblastoma, leukemia, neuroblastoma, head, neck, breast, pancreatic, prostate, renal, bone, testicular, ovarian, mesothelioma, cervical, gastrointestinal, urogenital, respiratory tract, hematopoietic, musculoskeletal, neuroendocrine, carcinoma, sarcoma, central nervous system, peripheral nervous system, lymphoma, brain, colon or bladder cancer.

25. The method of any one of claims 1-22, wherein the cancer is prostate cancer.

26. The method of claim 25, wherein the prostate cancer is further defined as castration-resistant prostate cancer.

27. The method of any one of claims 1-26, wherein the subject has a high serum level of sMIC.

28. The method of claim 27, wherein the high serum level of sMIC is at least 0.5 ng/mL.

29. The method of claim 27, wherein the serum level of sMIC in the subject decreases after administration of the anti-sMIC antibody and at least one immune checkpoint inhibitor.

30. The method of any one of claims 1-29, wherein the anti-sMIC antibody and at least one immune checkpoint inhibitor results in an increase in CD8⁺ T cells.

31. The method of claim 30, wherein the increase in CD8⁺ T cells is specific to the tumor draining lymph nodes, spleen, and/or tumor infiltrates.

32. The method of any one of claims 1-31, wherein the anti-sMIC antibody and at least one immune checkpoint inhibitor results in increased NKG2D expression on CD8⁺ T cells.

33. The method of any one of claims 1-32, wherein the anti-sMIC antibody and at least one immune checkpoint inhibitor results in an increase in effector memory-like CD44^{hi} CD8⁺ T cells in the spleen, dLN, and/or tumor infiltrates.

34. The method of any one of claims 1-33, wherein anti-sMIC antibody and at least one immune checkpoint inhibitor results in an increase in IFNγ production.

35. The method of any one of claims 1-34, further comprising the adoptive transfer of T cells.

36. The method of claim 35, wherein the T cells are CD8⁺ T cells.

37. The method of claim 36, wherein the CD8⁺ T cells are antigen-specific CD8⁺ T cells.

38. The method of claim 35, wherein the anti-sMIC antibody and at least one immune checkpoint inhibitor enhance the sustainability of the T cells in the subject.

39. The method of any one of claims 1-38, wherein the anti-sMIC antibody and at least one immune checkpoint inhibitor enhances dendritic cell activation.

40. The method of claim 39, wherein dendritic cell activation comprises an increase in the expression of co-stimulatory molecule CD80 and/or CD86.

41. The method of any one of claims 1-40, wherein the anti-sMIC antibody and at least one immune checkpoint inhibitor increases TCR clonality and/or repertoire diversity.

42. The method of any one of claims 1-41, wherein the anti-sMIC antibody and at least one immune checkpoint inhibitor increases the intracellular levels of CD3ζ in CD8⁺ T cells in the subject.

43. The method of any one of claims 1-42, wherein the anti-sMIC antibody and/or at least one immune checkpoint inhibitor is administered intratumorally, intraarterially, intravenously, intravascularly, intrapleuraly, intraperitoneally, intratracheally, intrathecally, intramuscularly, endoscopically, intralesionally, percutaneously, subcutaneously, regionally, stereotactically, or by direct injection or perfusion.

44. The method of any one of claims 1-43, wherein more than one checkpoint inhibitor is administered.

45. The method of any one of claims 1-44, further comprising administering at least one additional anticancer treatment.

46. The method of claim 45, wherein the at least one additional anticancer treatment is surgical therapy, chemotherapy, radiation therapy, hormonal therapy, immunotherapy, small molecule therapy, receptor kinase inhibitor therapy, anti-angiogenic therapy, cytokine therapy, cryotherapy or a biological therapy.

47. The method of claim 46, wherein the biological therapy is a monoclonal antibody, siRNA, miRNA, antisense oligonucleotide, ribozyme or gene therapy.

48. A pharmaceutical composition comprising an anti-sMIC antibody and at least one immune checkpoint inhibitor.

49. A composition comprising an effective amount of an anti-sMIC antibody or antigen-binding fragment thereof and at least one immune checkpoint inhibitor for use in the treatment of cancer in a subject.

50. The composition of claim 48 or claim 49, wherein the anti-sMIC antibody is a monoclonal antibody, a chimeric antibody, a CDR-grafted antibody, a humanized antibody, a Fab, a Fab′, a F(ab′)2, a Fv, or a scFv.

51. The composition of claim 48 or claim 49, wherein the anti-sMIC antibody is a monoclonal antibody.

52. The composition of claim 48 or claim 49, wherein the anti-sMIC antibody is a humanized monoclonal antibody.

53. The composition of claim 48 or claim 49, wherein the anti-sMIC antibody comprises a first VH CDR identical to SEQ ID NO: 1, a second VH CDR identical to SEQ ID NO: 2, a third VH CDR identical to SEQ ID NO: 3, a first VL CDR identical to SEQ ID NO: 4, a second VL CDR identical to SEQ ID NO: 5, and a third VL CDR identical to SEQ ID NO: 6.

54. The composition of claim 53, wherein the antibody is humanized.

55. The composition of claim 53, wherein the antibody is B10G5 monoclonal antibody.

56. The composition of claim 48 or claim 49, wherein the at least one immune checkpoint inhibitor is an anti-PD1 antibody or anti-CTLA4 antibody.

57. The composition of claim 48 or claim 49, wherein the at least immune checkpoint inhibitor is comprised in an oncolytic virus.

58. The composition of claim 57, wherein the oncolytic virus is myxoma virus.

59. The composition of claim 58, wherein the myxoma virus expresses soluble PD-1.

60. A method of predicting a response to an immune checkpoint inhibitor in a patient having a cancer comprising detecting a level of serum sMIC in a sample obtained from said patient, wherein if the serum sMIC level is low, then the patient is predicted to have a favorable response to the immune checkpoint inhibitor.

61. The method of claim 60, wherein a low serum sMIC level is further defined as less than 0.5 ng/mL.

62. The method of claim 60, wherein if the level of serum sMIC is high, then the patient is predicted to have adverse reaction to the immune checkpoint inhibitor.

63. The method of claim 62, wherein a high serum sMIC level identifies a patient in need of an anti-sMIC antibody in combination with the immune checkpoint inhibitor.

64. A method of treating colitis in a subject comprising administering an effective amount of an anti-sMIC antibody or antigen-binding fragment thereof to the subject.

65. The method of claim 64, wherein the anti-sMIC antibody or antigen-binding fragment thereof is a monoclonal antibody, a chimeric antibody, a CDR-grafted antibody, a humanized antibody, a Fab, a Fab′, a F(ab′)2, a Fv, or a scFv.

66. The method of claim 64, wherein the anti-sMIC antibody is a monoclonal antibody.

67. The method of claim 64, wherein the anti-sMIC antibody is a humanized antibody.

68. The method of claim 64, wherein the anti-sMIC antibody comprises a first VH CDR identical to SEQ ID NO: 1, a second VH CDR identical to SEQ ID NO: 2, a third VH CDR identical to SEQ ID NO: 3, a first VL CDR identical to SEQ ID NO: 4, a second VL CDR identical to SEQ ID NO: 5, and a third VL CDR identical to SEQ ID NO: 6.

69. The method of claim 68, wherein the antibody is humanized.

70. The method of claim 68, wherein the antibody is B10G5 monoclonal antibody.

71. The method of any one of claims 64-70, wherein the colitis is infectious colitis, inflammatory bowel disease (IBD), ischemic colitis, diversion colitis, chemical colitis, or microscopic colitis.

72. The method of claim 71, wherein the IBD is Crohn's disease or ulcerative colitis.

73. The method of any one of claims 64-72, wherein the subject has been determined to have a high serum level of sMIC.

74. The method of claim 73, wherein the high serum level of sMIC is at least 0.5 ng/mL.

75. The method of claim 73, further comprising determining the serum level of sMIC after administration of the anti-sMIC antibody.

76. The method of any one of claims 64-75, wherein the anti-sMIC antibody is administered intratumorally, intraarterially, intravenously, intravascularly, intrapleuraly, intraperitoneally, intratracheally, intrathecally, intramuscularly, endoscopically, intralesionally, percutaneously, subcutaneously, regionally, stereotactically, or by direct injection or perfusion.

77. The method of any one of claims 64-76, wherein the anti-sMIC antibody is administered subcutaneously.

78. The method of any one of claims 64-77, further comprising administering at least one additional therapy.

79. The method of claim 78, wherein the at least one additional therapy is an aminosalicylate, a corticosteroid, and/or an immunosuppressant.

80. The method of claim 79, wherein the aminosalicylate is sulfasalazine, mesalazine, balsalazide, and/or olsalazine.

81. The method of claim 79, wherein the corticosteroid is prednisone and/or hydrocortisone.

82. The method of claim 79, wherein the immunosuppressant is azathioprine, mercaptopurine, cyclosporingm infliximab, adalimumab, golimumab, and/or vedolizumab.
